(19) United States
(12) Patent Application Publication
(10) Pub. No.: US 2025/0312332 A1
(43) Pub. Date: Oct. 9, 2025

(54) TREATMENT OF MUSCLE FIBROSIS

(71) Applicants: University of Newcastle upon Tyne, Newcastle upon Tyne (GB); Fundació Institut de Recerca de l'hospital de la Santa Creu i Sant Pau, Barcelona (ES)

(72) Inventors: Esther FERNÁNDEZ-SIMÓN, Newcastle upon Tyne (GB); Xavier SUÁREZ-CALVET, Sabadell (ES); Jordi DÍAZ-MANERA, Newcastle upon Tyne (GB)

(21) Appl. No.: 18/855,996

(22) PCT Filed: Apr. 13, 2022
(86) PCT No.: PCT/GB2022/050924

§ 371 Date: Oct. 10, 2024

(51) Int. Cl. A61K 31/4725; A61K 31/551; A61K 38/45; A61P 21/00

(52) U.S. Cl. CPC A61K 31/4725; A61K 31/551; A61K 38/45; A61P 21/00

(57) ABSTRACT

The present invention relates to compounds for use in treating a disease or disorder associated with muscle fibrosis in a subject. In particular, the present invention relates to inhibitors of the RhoA/ROCK pathway for use in treating a disease or disorder associated with muscle fibrosis, like muscular dystrophy in a subject.

The present invention relates to compounds for use in treating a disease or disorder associated with muscle fibrosis in a subject. In particular, the present invention relates to inhibitors of the RhoA/ROCK pathway for use in treating a disease or disorder associated with muscle fibrosis, like muscular dystrophy in a subject.

### BACKGROUND

Fibrosis is a common key process in the degeneration of the skeletal muscle in patients with muscular dystrophies (MD), leading to muscle weakness, stiffness, contractures and permanent disability.

MD is a group of muscle disorders in which muscle fibers are unusually susceptible to damage. As a result, defects in muscle proteins accumulate, death of muscle cells and tissue occurs, and the musculoskeletal system of affected individuals becomes progressively weaker. Nine major types of muscular dystrophy have been identified: Duchenne, Becker, limb-girdle, congenital, facioscapulohumeral, myotonic, oculo-pharyngeal, distal and Emery-Dreifuss muscular dystrophy. Several muscular dystrophy-like conditions have also been identified.

In normal striated muscle, dystrophin associates with a large group of proteins known as the dystrophin glycoprotein complex (DGC). The DGC serves to stabilise the sarcolemma by making regularly spaced connections between the muscle fibre cytoskeleton and extracellular matrix—part of the costameric cell adhesion complex. At the core of this cell adhesion complex is the adhesion receptor dystroglycan, which binds laminin in the extracellular matrix and dystrophin on the cytoplasmic face. However, in a number of disorders, including the muscular dystrophies, generation of functional dystrophin protein and/or functional DGC is impaired.

Duchenne muscular dystrophy (DMD) is a severe muscle wasting disease that affects approximately 1 in 3,500 male births and for which there is currently no cure or effective treatment. Various molecular genetic approaches to combat DMD have been devised but are unlikely to address the need of all DMD sufferers: gene replacement using a number of delivery methods, compensatory gene upregulation and cell based therapies have all met with some success in the laboratory but have failed for a variety of reasons to translate to the clinic (Pichavant. C, et al. Mol Ther, 2011). More recently, however, significant successes have been achieved using exon skipping approaches to splice out mutated parts of the DMD gene and restore some functional dystrophin gene (Kinali. M, et al Lancet Neurol., 2009. 8:918). This is a rapidly developing area with phase II clinical trials of exon skipping in progress. These approaches provide real hope for the approximately 25% of DMD patients with no effective treatment. Clearly a therapeutic approach that could be effective for all DMD sufferers is still needed. Ideally, there is a need for a small molecule treatment which is simple to administer, does not require customization to a particular individual, and is well tolerated with a good safety profile. Such a treatment does not currently exist.

No specific treatments have thus far been approved to reduce fibrosis in muscular dystrophies. The only treatment approved for muscular dystrophies is corticosteroids, which are specifically used in DMD. Treatment with corticosteroids can slow down progression of disease by reducing inflammation in muscle tissue and thus preserving muscle strength. Delazacort and Prednisone are the main types of corticoids prescribed for DMD. The precise mechanism by which corticosteroids may increase muscle strength in DMD is not known but their potential beneficial effects include inhibition of muscle proteolysis, stimulation of myoblast proliferation, stabilization of muscle fibre membranes, increase in myogenic repair, anti-inflammatory/immunosuppressive effect, reduction of cytosolic calcium concentrations, up regulation of utrophin and differential regulation of genes in muscle fibres.

Fibro-adipogenic precursor cells (FAPs) are mesenchymal muscle-resident stem cells characterized by the expression of platelet-derived growth factor receptor alpha (PDGFRα). FAPs are the main cell type that is responsible for the fibrotic and fatty tissue expansion in muscular dystrophies. FAPs are activated upon acute injury, proliferate, and release components of the extracellular matrix (ECM) to serve as a scaffold for muscle regeneration. In healthy muscles, excessive FAPs are cleared by apoptosis, a process that is mediated by tumour necrosis factor alpha (TNF-α). However, in dystrophic muscles, continuous release of growth factors by M2 macrophages permanently activates FAP proliferation, which releases collagen-I (among other components of the ECM), leading to an expansion of fibrotic tissue.

The molecular pathways driving FAPs in muscular dystrophies are just starting to be understood. It is well-known that TGF-β promotes FAP proliferation, inhibits TNF-α mediated FAP apoptosis, and drives FAPs to differentiate into fibroblasts. In addition, platelet-derived growth factors (PDGFs) are involved in the muscle regeneration and degeneration process. PDGF-BB has been shown to activate satellite cell proliferation and chemotaxis and its receptor, PDGFRβ, is highly expressed in dystrophic muscle compared with healthy muscle. PDGF-AA binds to PDGFRα, a tyrosine kinase receptor, inducing receptor dimerization and autophosphorylation of the intracellular domain. Autophosphorylation of PDGFRα triggers signalling pathways such as Ras-MAPK, PI3K, or PLC-y, which are involved in different cellular responses including proliferation, cell differentiation, apoptosis inhibition, mobilization of intracellular calcium, or cell motility. It has previously been demonstrated that PFGF-AA activates fibroblast proliferation and migration and increases the release of components of the extracellular matrix. Previous studies have shown that PDGF-AA expression is higher in dystrophic muscle compared with healthy muscle and that PDGF-AA serum levels are increased in DMD patients. However, the pathways activated by PDGF-AA in FAPs in muscular dystrophies have not been elucidated.

Novel means for treating diseases or disorders associated with muscle fibrosis (such as muscular dystrophies) are urgently needed.

### BRIEF SUMMARY OF THE DISCLOSURE

The inventors used mass spectrometry to identify that the RhoA/ROCK pathway is activated by PDGF-AA in fibro-adipogenic precursor cells (FAPs) isolated from human skeletal muscle. Suprisingly, they showed that by inhibiting this pathway in a mouse model of muscular dystrophy, muscle fibrosis can be ameliorated. RhoA/ROCK pathway inhibitors can therefore advantageously be used to treat diseases or disorders associated with muscle fibrosis, such as muscular dystrophy.

The invention provides a method of treating a disease or disorder associated with muscle fibrosis in a subject, the method comprising administering a therapeutically effective amount of a RhoA/ROCK pathway inhibitor to the subject.

Suitably, the disease or disorder associated with muscle fibrosis may be a muscular dystrophy.

Suitably, the disease or disorder associated with muscle fibrosis may be a dystrophinopathy.

Suitably, the disease or disorder associated with muscle fibrosis may be selected from the group consisting of: Duchenne muscular dystrophy, Becker muscular dystrophy, recessive and dominant limb girdle muscular dystrophy, myotonic dystrophy type I, myotonic dystrophy type II, facio-scapulo-humeral muscular dystrophy, congenital muscular dystrophy, oculo-pharyngeal muscular dystrophy, Emery-Dreifuss muscular dystrophy, inclusion body myositis, distal myopathy with dystrophic changes and myofibrillar myopathy.

Suitably, the inhibitor may inhibit the RhoA/ROCK pathway by directly inhibiting ROCK1 and/or ROCK2.

Suitably, the inhibitor may be selected from the group consisting of: Fasudil, Y27632, Y39983, Wf-536, AR-13324, AR-12286, AMA0076, PG324, Azabenzimidazole-aminofurazans, DE-104, Olefins, Isoquinolines, Indazoles, pyridinealkene derivates, H-1152P, ROKα inhibitor, XD-4000, HMN-1152, 4-(1-aminoalkyl)-N-(4-pyridyl)cyclohexane-carboxamides, Quinazoline, Ripasudil, VAS-012, Ki-23095, BA-2017, BA-215, BA-285, BA-1037, BA-210, Rhostatin, ROCK-IN-1—preclinical, ROCK inhibitor-2, ROCK2-IN-5, ROCK2-IN-2, ROCK-IN-2 (Azaindole 1; TC-S 7001), Chroman 1, Cotosudil, SAR407899 hydrochloride, SAR407899, Hydroxyfasudil, Ripasudil, Ripasudil free base, Belumosudil mesylate, Belumosudil, Sovesudil, RKI-1447, H-1152, GSK269962A, Rho-Kinase-IN-1, SB-772077B dihydrochloride, HSD1590, BDP5290, ZINC00881524, Verosudil, LX7101, GSK-25, GSK180736A, CRT0066854, Y-27632, CMPD101, Thiazovivin, SR-3677, and GSK429286A or pharmaceutically acceptable salts, solvates or prodrugs thereof.

Suitably, the inhibitor may be a compound of formula (1) or a pharmaceutically acceptable salt, solvate or prodrug thereof:

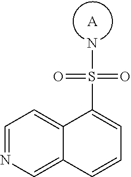

wherein:
A is a monocyclic 5- to 7-membered heterocycloalkyl ring optionally substituted with one or more R¹;
each R¹ is independently selected from C₁₋₆alkyl, hydroxy-C₁₋₆alkyl, halo-C₁₋₆alkyl and —C(O)OR²; and
R² is C₁₋₆ alkyl, and optionally wherein the compound of formula (1) is Fasudil.

Suitably, the inhibitor may be selected from the group consisting of: C3 exoenzyme, C3 Trans based, Rhosin, CCG-1423, CCG-203971, YS-49 monohydrate, YS-49, Cerivastatin sodium, Cerivastatin, Z62954982, Y16, MLS-573151, HA-100, HL07, DDO-5701, DDO-5713, DDO-5714, DDO-5715, DDO-5716, ML-7, MLCK18, and CT-04, or pharmaceutically acceptable salts, solvates or prodrugs thereof.

Suitably, the inhibitor may inhibit the RhoA/ROCK pathway by ribosylating RhoA proteins.

Suitably, the inhibitor may be C3 exoenzyme.

The invention also provides the use of a RhoA/ROCK pathway inhibitor for treating a disease or disorder associated with muscle fibrosis.

Suitably, the disease or disorder associated with muscle fibrosis may be a muscular dystrophy.

Suitably, the disease or disorder associated with muscle fibrosis may be a dystrophinopathy.

Suitably, the disease or disorder associated with muscle fibrosis may be selected from the group consisting of: Duchenne muscular dystrophy, Becker muscular dystrophy, recessive and dominant limb girdle muscular dystrophy, myotonic dystrophy type I, myotonic dystrophy type II, facio-scapulo-humeral muscular dystrophy, congenital muscular dystrophy, oculo-pharyngeal muscular dystrophy, Emery-Dreifuss muscular dystrophy, inclusion body myositis, distal myopathy with dystrophic changes and myofibrillar myopathy.

Suitably, the inhibitor may inhibit the RhoA/ROCK pathway by directly inhibiting ROCK1 and/or ROCK2.

Suitably, the inhibitor may be selected from the group consisting of: Fasudil, Y27632, Y39983, Wf-536, AR-13324, AR-12286, AMA0076, PG324, Azabenzimidazole-aminofurazans, DE-104, Olefins, Isoquinolines, Indazoles, pyridinealkene derivates, H-1152P, ROKα inhibitor, XD-4000, HMN-1152, 4-(1-aminoalkyl)-N-(4-pyridyl)cyclohexane-carboxamides, Quinazoline, Ripasudil, VAS-012, Ki-23095, BA-2017, BA-215, BA-285, BA-1037, BA-210, Rhostatin, ROCK-IN-1—preclinical, ROCK inhibitor-2, ROCK2-IN-5, ROCK2-IN-2, ROCK-IN-2 (Azaindole 1; TC-S 7001), Chroman 1, Cotosudil, SAR407899 hydrochloride, SAR407899, Hydroxyfasudil, Ripasudil, Ripasudil free base, Belumosudil mesylate, Belumosudil, Sovesudil, RKI-1447, H-1152, GSK269962A, Rho-Kinase-IN-1, SB-772077B dihydrochloride, HSD1590, BDP5290, ZINC00881524, Verosudil, LX7101, GSK-25, GSK180736A, CRT0066854, Y-27632, CMPD101, Thiazovivin, SR-3677, and GSK429286A, or pharmaceutically acceptable salts, solvates or prodrugs thereof.

Suitably, the inhibitor may be a compound of formula (1) or a pharmaceutically acceptable, salt, solvate or prodrug thereof:

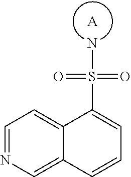

wherein:
A is a monocyclic 5- to 7-membered heterocycloalkyl ring optionally substituted with one or more R¹;
each R¹ is independently selected from C₁₋₆alkyl, hydroxy-C₁₋₆alkyl, halo-C₁₋₆alkyl and —C(O)OR²; and
R² is C₁₋₆ alkyl, and optionally wherein the compound of formula (1) is Fasudil.

Suitably, the inhibitor may be selected from the group consisting of C3 exoenzyme, C3 Trans based, Rhosin, CCG-1423, CCG-203971, YS-49 monohydrate, YS-49, Cerivastatin sodium, Cerivastatin, Z62954982, Y16, MLS-573151, HA-100, HL07, DDO-5701, DDO-5713, DDO-5714, DDO-5715, DDO-5716, ML-7, MLCK18, CT-04, or pharmaceutically acceptable salts, solvates or prodrugs thereof.

Suitably, the inhibitor may inhibit the RhoA/ROCK pathway by ribosylating RhoA proteins.

Suitably, the inhibitor may be C3 exoenzyme.

Various aspects of the invention are described in further detail below.

### BRIEF DESCRIPTION OF THE FIGURES

Embodiments of the invention are further described hereinafter with reference to the accompanying figures:

FIGS. 1A-H A) Representative images of the muscle derived cells from DMD patients. CD56− were stained with desmin and TE-7 markers to validate the purification step. Scale bar: 100 μm B) Quantification of PDGFRα+ expression area in muscle tissue obtained from healthy controls (n=2) and DMD patients (n=2). C) Representative images of the muscle biopsies from healthy control and DMD patient stained with PDGFRα antibody. Scale bar: 100 μm. D) Flow cytometry isotype control staining and analysis of the PDGFRα in the CD56-isolated cells showing positivity for the receptor. D) Experimental plan to screen. F) Representative WB of total protein bands measured with the Revert 700 and used as a loading control for ArhGEF2 protein quantification. G) Representative in-cell western image showing the total cell number signal stained with TO-PRO-3. H) Viability determination using the PrestoBlue cell viability assay to compare the effect of PDGF-AA, C3 and fasudil on FAPs at 30 min, 24 h, 48 h and 72 h. Data are represented as the mean of 3 replicates±standard error of the mean. Results were statistically analysed using one-way ANOVA followed by Tukey post hoc test.

FIGS. 2A-E show the proteome analysis of PDGF-AA treated DMS FAPs. (A) Volcano plot representing the total proteome. Data are presented as the protein abundance changes of PDGF-AA treated FAPs relative to untreated FAPs. Significantly differentially expressed proteins (Student's t-test, P≤0.05) are highlighted in light grey. (B-D) Proteomap of up-regulated proteins after PDGF-AA treatment. The proteomic map visualizes the composition of the proteomes in terms of abundance and function of the proteins. Each protein is represented by a polygon, and the area of each polygon reflects the abundance of the proteins (calculated with fold-change). Functionally related proteins appear in adjacent regions. The three panels represent three hierarchy levels, being the first one the individual protein detected (B), the second one shows proteins grouped into pathways (C), and the last one shows the functional category associated to a group of proteins (D). (E) Up-regulated proteins shown in proteomaps were also represented in heatmap showing the results of Reactome database.

FIGS. 3A-F show that the RhoA pathway is upregulated in DMD and PDGF-AA activates it. (A) Molecular pathway of RhoA/ROCK2 signalling downstream of ArhGEF2 which catalyses the replacement of RhoA-GDP to RhoA-GTP, controlling RhoA activation. RhoA bound to GTP activates the rho-associated coiled coil-forming protein kinase (ROCK), a serine/threonine kinase that regulates actin filament remodelling trough light chain myosin (MLC) phosphorylation. (B) Quantification of WB bands of ArhGEF2 relative to total protein detection and representative bands of ArhGEF2 expression in muscle tissue obtained from healthy controls (n=2) and DMD patients (n=2). Total protein detection was used as loading control signal (FIG. 1D). (C) Optical density at 490 nm indicating RhoA activity relative to total RhoA protein detected by WB. (D) Representative images of p-MLC staining using fluorescence microscopy. Scale bar: 50 μm. (E) Quantification of p-MLC intensity of each cell after different treatments. (F) Representative images FAPs stained with phalloidin after different treatments. Scale bar: 50 μm. C-: Untreated FAPs. Data are represented as the mean of three replicates±standard error of the mean. Results were statistically analysed using one-way ANOVA followed by Tukey post hoc test. Statistical significance was set at P<0.05. **P<0.01; ***P<0.001.

FIGS. 4A-D show the effects of C3 exoenzyme, fasudil, and PDGF-AA on FAPs from DMD patients. (A) FAP proliferation was analysed after 48 and 72 h. (B) FAP migration was analysed after 48 and 72 h. (C) Representative images of migration assay at both 48 and 72 h. Scale bar: 200 μm. (D) The amount of collagen delivered to the supernatant was analysed after 4 days treatment. C-: Untreated FAPs. Data are represented as the mean of three replicates±standard error of the mean. Results were statistically analysed using one-way ANOVA followed by Tukey post hoc test. Statistical significance was set at P<0.05. *P<0.05; **P<0.01; ***P<0.001.

FIGS. 5A-C A) Representative immunofluorescence images of PDGF-AA expression in quadriceps sample from dba/2J-WT and dba/2J-mdx. Scale bar: 100 μm. B) Effect of fasudil treatment in the dba/2J-mdx mice. A) Weight measurement after 6 weeks treatment with fasudil in dba-2J WT mice, untreated dba/2J-mdx and fasudil treated dba/2J-mdx. C) Representative immunofluorescence images of collagen I expression in the heart. Scale bar: 100 μm. and quantification of collagen I expression area in the right and left ventricle of the heart. D) Percentage and mean intense fluorescence (MFI) of CD209, CD80 and DR markers analysed by flow cytometry. Data are represented as the mean of the groups analysed±standard error of the mean. Results were statistically analysed using one-way ANOVA followed by Tukey post hoc test. Statistical significance was set at p<0.05. *p<0.05; **p<0.01; ***p<0.001.

FIGS. 6A-F A) Relative gene expression of ROCK2, LIMK2 and FHOD1 in quadriceps in dba-2J WT mice, untreated dba/2J-mdx and fasudil treated dba/2J-mdx. B) Quantification of ROCK activity in quadriceps in dba-2J WT mice, untreated dba/2J-mdx and fasudil treated dba/2J-mdx. C) Representative immunofluorescence images of LIMK2 expression in quadriceps. Scale bar: 100 μm. D) Quantification of LIMK2 expression area in quadriceps. E) Representative immunofluorescence images of MLC2 expression in quadriceps. Scale bar: 100 μm. D) Quantification of MLC2 expression area in quadriceps. Data are represented as the mean of the groups analysed±standard error of the mean. Results were statistically analysed using one-way ANOVA followed by Tukey post hoc test. Statistical significance was set at p<0.05. *p<0.05; **p<0.01; ***p<0.001.

FIGS. 7A-I show the effect of fasudil treatment in the dba/2J-mdx mice. (A) Grip strength test after 6 weeks treatment with fasudil in dba-2J WT mice, untreated dba/2J-mdx, and fasudil treated dba/2J-mdx. Results are relative to weight of animals. (B) Quantification of collagen I expression area in quadriceps. Scale bar: 200 μm. (C) Quantification of muscle fibre size mean and frequency in dba-2J WT mice, untreated dba/2J-mdx, and fasudil treated dba/2Jmdx. (D) Representative immunofluorescence images of collagen I expression in muscle samples. Scale bar: 200 μm. (E) Quantification of PDGFRα+ expression area in quadriceps. (F) Representative immunofluorescence images of PDGFRα+ (light grey arrows), F4/80 (white arrows) expression in muscle samples. ECM is stained with wheat germ agglutinin (WGA) in green. Scale bar: 200 μm. (G) Quantification of macrophages expression area in quadriceps. (H) Quantification of results obtained with flow cytometry to analyse the F4/80 macrophages (Mo) accumulated in quadriceps and tibial anterior obtained from mice. Data are represented as counts of F4/80 relative to the weight of quadriceps and tibial anterior. (I) Percentage of total macrophages and mean fluorescence intensity (MFI) of CD163 and CD206 markers were analysed by flow cytometry. Data are represented as the mean of the groups analysed±standard error of the mean. Results were statistically analysed using one-way ANOVA followed by Tukey post hoc test. Statistical significance was set at P<0.05. *P<0.05; **P<0.01; ***P<0.001.

FIGS. 8A-E A) Representative immunofluorescence images of T cells by CD3 (light grey arrows), neutrophils by Ly6G (white arrows) and B cells by CD19 cells. Scale bar: 100 μm. B) Quantification of T cells (CD3+ cells) in each section in quadriceps. C) Quantification of neutrophils (Ly6G+ cells) in each section in quadriceps. D) Quantification of B cells (CD19+ cells) in each section in quadriceps. Results were statistically analysed using one-way ANOVA followed by Tukey post hoc test. Statistical significance was set at p<0.05. **p<0.01; ***p<0.001. E) Heatmap representation of cytokine levels measured in 3 muscles from each group: dba/2J-WT (n=3), dba/2J-mdx (n=3) and dba/2J-mdx treated with fasudil (n=3).

### DETAILED DESCRIPTION

The present invention relates to compounds for use in treating a disease or disorder associated with muscle fibrosis in a subject. In particular, the present invention relates to inhibitors of the RhoA/ROCK pathway for use in treating a disease or disorder associated with muscle fibrosis.

The authors have surprisingly identified that the RhoA/ROCK pathway is activated in muscle fibrotic processes and that by inhibiting this pathway muscle fibrosis can be ameliorated.

### Treatment of a Subject

According to a first aspect a method of treating a disease or disorder associated with muscle fibrosis in a subject is provided, the method comprising administering a therapeutically effective amount of a RhoA/ROCK pathway inhibitor to the subject.

Inhibiting the RhoA/ROCK pathway blocks the activation of FAPs. This inhibits/slows down the fibrotic process as the FAPs can no longer proliferate, migrate and reorganize actin.

“Fibrosis” refers to the thickening and scarring of connective tissue and is well known to a person skilled in the art. The term “muscle” relates to any type of muscle in a body like skeletal (striated), smooth and cardiac muscles. As used herein the terms “muscle fibrosis” or “muscle fibrotic process” therefore refer to any kind of fibrosis affecting the muscle. Muscle fibrosis may muscle function, negatively affect muscle regeneration after injury or increase muscle susceptibility to re-injury. A “disease or disorder associated with muscle fibrosis” refers to a disease or disorder in which muscle fibrosis occurs (e.g, wherein muscle fibrosis is a pathological symptom or manifestation of the disease or disorder). The phrase “a disorder associated with muscle fibrosis” therefore encompasses any disease or disorder resulting directly or indirectly from and/or completely or partially from muscle fibrosis. Muscle fibrosis can be the origin of the disease or disorder but can also be a symptom appearing after onset of the disease or disorder. Appropriate diseases or disorders are well known to a person of skill in the art. Suitable diseases or disorders associated with muscle fibrosis are described in detail elsewhere herein.

The RhoA/ROCK pathway inhibitors described herein may be used in treating any disease or disorder associated with muscle fibrosis in a subject. In one example, the disease or disorder associated with muscle fibrosis is muscular dystrophy.

Muscular Dystrophy (MD) is a group of muscle disorders in which muscle fibers are unusually susceptible to damage. As a result, defects in muscle proteins accumulate, death of muscle cells and tissue occurs, and the musculoskeletal system of affected individuals becomes progressively weaker.

Muscle tissue from patients with muscular dystrophies is characterized by the loss of muscle tissue and their replacement by fat and fibrous tissue, leading to permanent weakness and disability. Symptoms of MD therefore include muscle weakness or degeneration, calf hypertrophy, reduced myofibre integrity, elevated serum creatine kinase levels, loss of dystrophin and dystrophin associated proteins, and central nucleation of muscle fibres. Methods for assessing and/or identifying muscle weakness or degeneration, calf hypertrophy, reduced myofibre integrity, elevated serum creatine kinase levels, loss of dystrophin and dystrophin associated proteins, and central nucleation of muscle fibres are well known. By way of example, but without limitation, loss of dystrophin and dystrophin associated proteins may be assessed at a histological or a molecular (e.g. using PCR) level.

In one example, the disease or disorder associated with muscle fibrosis is dystrophinopathy.

Dystrophinopathy refers to a spectrum of diseases due to mutations in the DMD gene, which encodes the dystrophin protein found in muscle. The severe end of the spectrum includes Duchenne muscular dystrophy (DMD), Becker muscular dystrophy (BMD), and DMD-associated dilated cardiomyopathy. The mild end of the spectrum includes asymptomatic increases in serum creatine kinase and muscle cramps with myoglobinuria. Because dystrophin is located on the X chromosome, dystrophinopathy mainly affects males, whereas females range from being carriers, to having delayed-onset and mild disease, to having severe DMD.

In one example, the dystrophinopathy is Duchenne muscular dystrophy (DMD), Becker muscular dystrophy (BMD). These diseases are closely related, with similar treatment options.

In one example, the disease or disorder associated with muscle fibrosis is selected from the group consisting of: Duchenne muscular dystrophy, Becker muscular dystrophy, recessive and dominant limb girdle muscular dystrophy, myotonic dystrophy type I, myotonic dystrophy type II, facio-scapulo-humeral muscular dystrophy, congenital muscular dystrophy, oculo-pharyngeal muscular dystrophy, Emery-Dreifuss muscular dystrophy, inclusion body myositis, distal myopathy with dystrophic changes and myofibrillar myopathy.

As used herein, the terms “Limb Girdle Muscular Dystrophy” and “LGMD” are intended to cover all muscle dystrophies included in the current classification published in 2018 (Straub et al, Neuromuscular Disorders DOI: 10.1016/j.nmd.2018.05.007) including autosomal dominant LGMD (LGMD-D) 1 to 4 and autosomal recessive LGMD (LGMD-R 1 to 20). In one example, the Limb Girdle Muscular Dystrophy may be selected from the group consisting of: Limb Girdle Muscular Dystrophy R3, Limb Girdle Muscular Dystrophy R4, Limb Girdle Muscular Dystrophy R6, and Limb Girdle Muscular Dystrophy R7.

As used herein, the terms “Congenital Muscular Dystrophy” and “CMD” are intended to include Laminin-α2-deficient CMD (MDC1A), Ullrich congenital muscular dystrophy (UCMDs 1, 2 and 3), Walker-Warburg syndrome (WWS), Muscle-eye-brain disease (MEB), Fukuyama CMD (FCMD). CMD plus secondary laminin deficiency 1 and 2 (MDC1B and MDC1C), CMD with meta retardation and pachygyria (MDC1D) and rigid spine with muscular dystrophy type 1 (RSMD). In one example, the Congenital Muscular Dystrophy may be selected from the group consisting of MDC1A, MDC1B, MDC1D, Fukuyama CMD (FCMD), Muscle eye brain disease (MEB) and Walker Warburg Syndrome (WWS).

The term “RhoA” is the abbreviation for “Ras homolog gene family, member A”. It refers to a small GTPase protein in the Rho family. In humans, RhoA is encoded by the gene RHOA, is located on chromosome 3 and has an effector domain, four exons, a hypervariable region and a CAAX box motif (C: Cys; A: aliphatic residue; X: any residue). The N-terminus region of RhoA contains two switch regions, Switch I and Switch II, that have characteristic folding. The conformations of these switches are modified following the activation or inactivation of the RhoA protein. The C-terminus of RhoA is essential for correct localization of the protein. RhoA protein is expressed in all tissues including normal human tissues, embryonic tissues and stem cells. RhoA localizes predominantly in the plasma membrane and cytoplasm, as well as near the cell-cell contacts and cell projections. RhoA plays an important role in multiple cellular processes such as cell growth, transformation, and cytoskeleton regulation.

The term “ROCK” is the abbreviation for “Rho-associated protein kinase”. ROCK is a downstream effector of RhoA, which exists in two isoforms, ROCK1 and ROCK2. ROCK is a kinase belonging to the AGC (PKA/PKG/PKC) family of serine-threonine specific protein kinases. It is mainly involved in regulating the shape and movement of cells by acting on the cytoskeleton.

ROCKs (ROCK1 and ROCK2) occur in mammals (human, rat, mouse, cow), zebrafish, Xenopus, invertebrates (C. elegans, mosquito, Drosophila) and chickens. Human ROCK1 has a molecular mass of 158 kDa and is a major downstream effector of the small GTPase RhoA. Mammalian ROCK has a kinase domain, a coiled-coil region and a Pleckstrin homology (PH) domain, which reduces the kinase activity of ROCKs by an autoinhibitory intramolecular fold when RhoA-GTP is not present.

The Rho/ROCK signalling pathway is an important signal transduction system that is critically involved in cell growth, differentiation, migration and development.

The term “inhibitor” as used herein refers to any compound that reduces, abolishes, prevents, blocks, suppress, slows, or interferes the signalling of the RhoA/ROCK pathway, either directly or indirectly. Several inhibitors of the RhoA/ROCK pathway are known. Suitable examples are listed elsewhere herein. Inhibition may be reversible or irreversible.

As would be clear to a person skilled in the art, an inhibitor may function at the level of the target gene, transcript or protein. In some examples, the inhibitor may reduce RhoA/ROCK activity by at least 5%, 10%, 15%, 20%, 25%, 30%, 35%, 40%, 45%, 50%, 55%, 60%, 65%, 70%, 75%, 80%, 85%, 90%, 95%, or 100% as compared to the activity of a control (e.g., activity in the absence of the inhibitor).

An inhibitor can be an agent (e.g. an inhibitory nucleic acid, a binding molecule (e.g. a small molecule or an antibody), or peptide) that inhibits RhoA/ROCK function. A person of skill in the art will be able to readily identify suitable RhoA/ROCK pathway inhibitors using known methods in the art (e.g. based on assaying the effect of potential inhibitors on known RhoA/ROCK activity).

In some examples, the inhibitor inhibits, reduces, slows, halts, blocks, supresses, abolishes and/or prevents ROCK activity. In another example, the inhibitor inhibits, reduces, slows, halts, blocks, supresses, abolishes and/or prevents the RhoA activity.

In a particular example, the inhibitor is a direct ROCK inhibitor. In another example, the inhibitor is a direct RhoA inhibitor. A “direct inhibitor”, as used herein, refers to an inhibitor that directly targets the target gene, transcript or protein. In the context of ROCK, a direct inhibitor directly inhibits the ROCK protein, a ROCK transcript and/or the ROCK gene, thereby reducing ROCK protein activity and/or expression. In the context of RhoA, a direct inhibitor directly inhibits the RhoA protein, a RhoA transcript and/or the RhoA gene, thereby reducing RhoA protein activity and/or expression.

Accordingly, in some examples, the inhibitor directly targets the ROCK gene, RNA transcript or protein. For example, the inhibitor may directly bind to the ROCK gene, RNA transcript or protein. In some examples, the inhibitor directly targets the RhoA gene, RNA transcript or protein. For example, the inhibitor may directly bind to the RhoA gene, RNA transcript or protein.

Any suitable ROCK inhibitor and/or RhoA inhibitor may be used. In a particular example, the ROCK inhibitor and/or RhoA inhibitor may be selected from the group consisting of: an inhibitory nucleic acid, a binding molecule (e.g. a small molecule or an antibody (including functional fragments thereof)) and a peptide. Suitable examples of inhibitory nucleic acids, binding molecules (e.g. small molecules, or antibodies (including functional fragments thereof)) or peptides would be readily identifiable to a person of skill in the art.

In some examples, the inhibitor may be an “inhibitory nucleic acid” whose presence in a cell causes the degradation of or inhibits the function, transcription, or translation of its target gene in a sequence-specific manner. Exemplary inhibitory nucleic acids include aptamers, siRNA, microRNA-adapted shRNA, shRNA, precursor microRNA (pre-miRNA), pri-miRNA, miRNA, amiRNA, interfering RNA or RNAi, dsRNA, ribozymes, antisense oligonucleotides (ASO), and DNA expression cassettes encoding said inhibitory nucleic acids.

In some examples, the inhibitor is a binding molecule. Preferably, the binding molecule binds to ROCK or RhoA and inhibits its activity (for example it inhibits one or more of the activities for ROCK or RhoA described elsewhere herein).

An example of a binding molecule is a small molecule. Binding molecules also include antibodies as well as non-immunoglobulin binding agents, such as phage display-derived peptide binders, and antibody mimics, e.g., affibodies, tetranectins (CTLDs), adnectins (monobodies), anticalins, DARPins (ankyrins), avimers, iMabs, microbodies, peptide aptamers, Kunitz domains, aptamers and affilins. The term “antibody” includes, for example, both naturally occurring and non-naturally occurring antibodies, polyclonal and monoclonal antibodies, chimeric antibodies and wholly synthetic antibodies and fragments thereof, such as, for example, the Fab′, F(ab′)2, Fv or Fab fragments, or other antigen recognizing immunoglobulin fragments. Antibodies which bind a particular epitope can be generated by methods known in the art. For example, polyclonal antibodies can be made by the conventional method of immunizing a mammal (e.g., rabbits, mice, rats, sheep, goats). Polyclonal antibodies are then contained in the sera of the immunized animals and can be isolated using standard procedures (e.g., affinity chromatography, immunoprecipitation, size exclusion chromatography, and ion exchange chromatography). Monoclonal antibodies can be made by the conventional method of immunization of a mammal, followed by isolation of plasma B cells producing the monoclonal antibodies of interest and fusion with a myeloma cell (see, e.g., Mishell, et al., 1980). Screening for recognition of the epitope can be performed using standard immunoassay methods including ELISA techniques, radioimmunoassays, immunofluorescence, immunohistochemistry, and Western blotting. In vitro methods of antibody selection, such as antibody phage display, may also be used to generate antibodies. Preferably, a nuclear localization signal is added to the antibody in order to increase localization to the nucleus.

In some examples, the activity and/or expression of a target protein (e.g. ROCK or RhoA) in a cell may be inhibited, reduced, slowed, halted, blocked, suppressed, abolished and/or prevented by introduction of a mutation that disrupts the target gene (e.g. by introduction of a mutation that disrupts the ROCK gene or the RhoA gene). As would be clear to a skilled person, such a mutation may decrease expression of the protein encoded by the target gene (e.g. ROCK or RhoA), abrogate expression of the gene entirely, or render the gene product non-functional. Accordingly, in some examples, the mutation may be a loss of function mutation. In some examples, the mutation may be a point mutation, an insertion, a substitution or a deletion. In some examples, both alleles of the target gene are mutated. In some examples, the mutation may be located in a coding region (e.g., in an exon of ROCK or RhoA) and/or in a non-coding region of the target gene (e.g. in the promoter region of ROCK or RhoA).

Mutation of the ROCK gene and/or RhoA gene may be accomplished by methods well known in the art, including gene editing techniques. For example, a mutation may be introduced into the ROCK gene and/or the RhoA gene using a targeted genome editing technique (e.g. using targeted genome editing construct(s)). For example, a mutation may be introduced into the ROCK gene and/or the RhoA gene using zinc-finger nucleases (ZFNs), transcription activator-like effector nucleases (TALENs), clustered regularly interspaced short palindromic repeat (CRISPR) nucleases (e.g. RNA-guided DNA endonuclease Cas9 (or variants thereof)) or meganucleases.

In one example, the inhibitor inhibits the RhoA/ROCK pathway by directly inhibiting ROCK1 and/or ROCK2. In this context, “directly inhibiting ROCK” may refer to inhibiting ROCK's ability to phosphorylate myosin light chain 2 (p-MLC2), which when phosphorylated would lead to a cytoskeletal rearrangement (see FIG. 3A).

The ROCK inhibitor can either be a “pan-ROCK inhibitor”, which is a selective inhibitor of both ROCK1 and ROCK2 isoforms, or it can be a ROCK1 or ROCK2 specific inhibitor. For example, a ROCK1 specific inhibitor can inhibit ROCK1 but not ROCK2 whereas a ROCK2 specific inhibitor can inhibit ROCK2 but not ROCK1. A pan-ROCK inhibitor can inhibit both isoforms with either equal potency and no selective effects or it can inhibit both but has a higher potency for either ROCK1 or ROCK2. For example, a pan-ROCK inhibitor with a higher potency for ROCK1 can inhibit both isoforms but inhibits ROCK1 with a higher efficiency. ROCK2-IN-2 is one example of a selective ROCK2 inhibitor and GSK429286A is an example of a selective ROCK1 inhibitor.

In one example, the ROCK inhibitor is a pan-ROCK inhibitor, or a ROCK2 inhibitor. In one example, the ROCK inhibitor is a direct pan-ROCK inhibitor or a direct ROCK2 inhibitor.

In one example, the inhibitor is a RhoA/ROCK2 pathway inhibitor.

In one example, the inhibitor is selected from the group consisting of: Fasudil, Fasudil hydrochloride, Y27632, Y39983, Wf-536, AR-13324, AR-12286, AMA0076, PG324, Azabenzimidazole-aminofurazans, DE-104, Olefins, Isoquinolines, Indazoles, pyridinealkene H-1152P, ROKα inhibitor, XD-4000, HMN-1152, 4-(1-aminoalkyl)-N-(4-derivates, pyridyl)cyclohexane-carboxamides, Quinazoline, Ripasudil, Ripasudil hydrochloride, Ripasudil hydrochloride hydrate, VAS-012, Ki-23095, BA-2017, BA-215, BA-285, BA-1037, BA-210, Rhostatin, ROCK-IN-1—preclinical, ROCK inhibitor-2, ROCK2-IN-5, ROCK2-IN-2, ROCK-IN-2 (Azaindole 1; TC-S 7001), Chroman 1, Chroman 1 dihydrochloride, Cotosudil, SAR407899 hydrochloride, SAR407899, Hydroxyfasudil, Hydroxyfasudil hydrochloride, Ripasudil, Ripasudil free base, Belumosudil mesylate, Belumosudil, Sovesudil, Sovesudil hydrochloride, RKI-1447, RKI-1447 dihydrochloride, H-1152 dihydrochloride, H-1152, GSK269962A, GSK269962A hydrochloride, Rho-Kinase-IN-1, SB-772077B dihydrochloride, HSD1590, BDP5290, ZINC00881524, Verosudil, LX7101, GSK-25, GSK180736A, CRT0066854, CRT0066854 hydrochloride, Y-27632 dihydrochloride, Y-27632, CMPD101, Thiazovivin, SR-3677, and GSK429286A. These are examples of inhibitors that directly inhibit ROCK1 and/or ROCK2.

In one example, the inhibitor is a compound of formula (1) or a pharmaceutically acceptable salt, solvate or prodrug thereof:

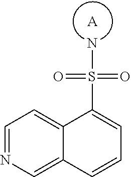

wherein:
A is a monocyclic 5- to 7-membered heterocycloalkyl ring optionally substituted with one or more R¹;
each R¹ is independently selected from C₁₋₆alkyl, hydroxy-C₁₋₆alkyl, halo-C₁₋₆alkyl and —C(O)OR²; and
R² is C₁₋₆ alkyl.

Compounds of formula (1) can be prepared using the methods described in H. Li et al, Journal of Heterocyclic Chemistry, 56 (1), 2019, pp. 260-267.

The term “compound of formula (1)” encompasses all stereoisomers, i.e. enantiomers (e.g. R and S enantiomers) and diastereomers, of such compounds and all salts thereof, in substantially pure form and/or any mixtures of the foregoing in any ratio. Thus, any reference to a compound of formula (1) refers to an enantiomer or a diastereomer thereof.

The monocyclic 5- to 7-membered heterocycloalkyl ring is typically fully saturated (e.g. the monocyclic 5- to 7-membered heterocycloalkyl ring does not have any double or triple bonds).

The monocyclic 5- to 7-membered heterocycloalkyl ring may include a total of one, two, three or four heteroatoms. It is preferred that the monocyclic 5- to 7-membered heterocycloalkyl ring has a total of one or two heteroatoms.

One of the heteroatoms, as shown in formula (1), is a nitrogen atom. Each of the other heteroatoms may be selected from an oxygen atom and a nitrogen atom, more preferably each of the other heteroatoms is a nitrogen atom.

It is preferred that the monocyclic 5- to 7-membered heterocycloalkyl ring is a 5- or 7-membered heterocycloalkyl ring having a total of one or two heteroatoms.

Typically, A is selected from:

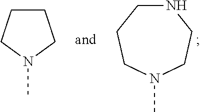

optionally substituted with one or more R¹. The dashed bond indicates the point of attachment of the nitrogen atom to the sulfur atom in formula (1).

In general, the halo-C₁₋₆alkyl is preferably chloro C₁₋₆alkyl (e.g. the halo group is a chloro group).

Each R¹ is preferably independently selected from hydroxy-C₁₋₆alkyl and halo-C₁₋₆alkyl, more preferably hydroxy-C₁₋₂alkyl and halo-C₁₋₂alkyl.

The compound of formula (1) may be selected from:

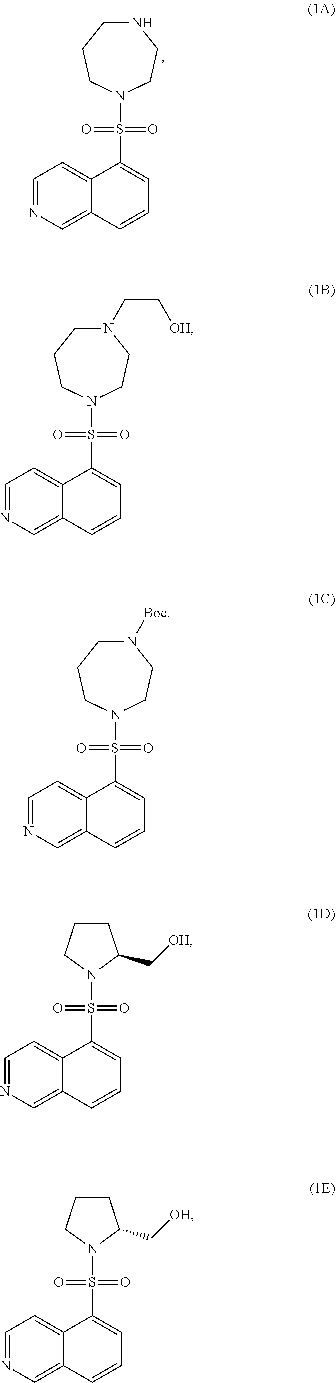 [SMILES: O=S(=O)(c1cccc2cnccc12)N1CCCNCC1 ; O=S(=O)(c1cccc2cnccc12)N1CCCN(CCO)CC1 ; CC(C)(C)OC(=O)N1CCCN(S(=O)(=O)c2cccc3cnccc23)CC1 ; O=S(=O)(c1cccc2cnccc12)N1CCC[C@H]1CO ; O=S(=O)(c1cccc2cnccc12)N1CCC[C@@H]1CO]

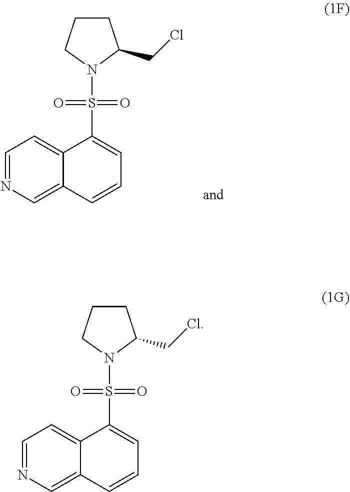 [SMILES: O=S(=O)(c1cccc2cnccc12)N1CCC[C@H]1CCl ; O=S(=O)(c1cccc2cnccc12)N1CCC[C@@H]1CCl]

or is a pharmaceutically acceptable salt, solvate or prodrug thereof. The group “Boc” refers to a tert-butyloxycarbonyl group.

It is preferred that the compound of formula (1) is a compound selected from (1A), (1B) and (1D) to (1G) above, or is a pharmaceutically acceptable salt, solvate or prodrug thereof.

Compound (1A) is known as Fasudil (IUPAC name is 5-(1,4-diazepan-1-ylsulfonyl)isoquinoline).

It is preferred that the compound of formula (1) is Fasudil (CAS No.: 103745-39-7) or Fasudil hydrochloride (CAS No.: 105628-07-7).

Fasudil or Fasudil hydrochloride is a commercially available vasodilator used to treat cerebral vasospasm. It is also useful for pulmonar hypertension. Fasudil inhibits the RhoA/ROCK pathway by blocking ROCK, which phosphorylates myosin light chain 2 (p-MLC2). Fasudil targets the ATP-dependent kinase domain of either ROCK1 and ROCK2 with equal potency and without selective effects.

Fasudil hydrochloride has the structural formula:

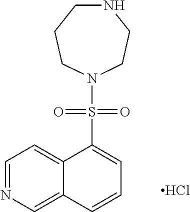 [SMILES: O=S(=O)(c1cccc2cnccc12)N1CCCNCC1 ; Cl]

In one example, the inhibitor is Fasudil, or a pharmaceutically acceptable salt, solvate or prodrug thereof.

Y27632 (CAS 146986-50-7) is an orally active, ATP-competitive inhibitor of ROCK-I and ROCK-II. Y-27632 attenuates Doxorubicin-induced apoptosis of human cardiac stem cells. Y-27632 also suppresses dissociation-induced apoptosis of murine prostate stem/progenitor cells. Y-27632 primes human induced pluripotent stem cells (hIPSCs) to selectively differentiate towards mesendodermal lineage via epithelial-mesenchymal transition-like modulation. Y-27632 has the structural formula:

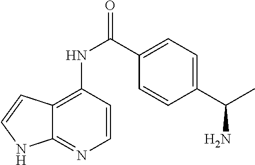 [SMILES: C[C@@H](N)c1ccc(C(=O)Nc2ccnc3[nH]ccc23)cc1]

In one example, the inhibitor is Y-27632 or a pharmaceutically acceptable salt, solvate or prodrug thereof.

Y39983 (CAS: 199433-58-4), also known as Y-33075 and RKI-983 and its derivative Y-33075 dihydrochloride are selective ROCK inhibitors derived from Y-27632, and is more potent than Y-27632, with an IC50 of 3.6 nM. Y-39983 has the following formula:

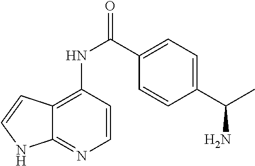 [SMILES: C[C@@H](N)c1ccc(C(=O)Nc2ccnc3[nH]ccc23)cc1]

In one example, the inhibitor is Y-39983 or a pharmaceutically acceptable salt, solvate or prodrug thereof.

Y-33075 dihydrochloride (CAS No.: 173897-44-4) has the structural formula:

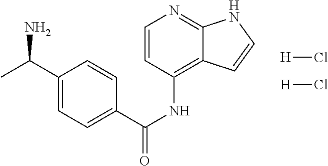

In one example, the inhibitor is Y-33075 or a pharmaceutically acceptable salt, solvate or prodrug thereof. The inhibitor may be Y-33075 dihydrochloride.

ROCK2 Selective Inhibitor (RXC007) is an orally available, highly selective small molecule inhibitor that targets Rho Associated Coiled-Coil Containing Protein Kinase 2 (ROCK2). RXC007 is currently in a Phase 1 clinical study in healthy volunteers, with IPF being targeted as the first indication for clinical development.

Wf-536 (CAS539857-64-2) [(+)-(R)-4-(1-aminoethyl)-N-(4-pyridyl)benzamide monohydrochloride] is an inhibitor of ROCK that effectively reduced in vitro invasion and in vivo pulmonary metastasis of B16 melanoma. Wf-536 has the following formula:

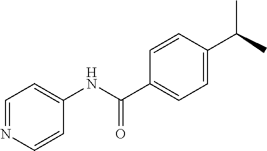

In one example, the inhibitor is Wf-536 or a pharmaceutically acceptable salt, solvate or prodrug thereof.

Belumosudil (CAS 911417-87-3) also known as KD025 or SLx-2119, is a selective inhibitor of ROCK2 with IC50s of 105 nM and 24 μM for ROCK2 and ROCK1, respectively with anti-fibrotic properties. Belumosudil mesylate is a derivative of belumosudil (CAS 2109704-99-4). Belumosudil induces significant down-regulations of Tsp-1 and CTGF mRNA levels in PASMC. Moreover, belumosudil dose-dependently reduces infarct volume after transient middle cerebral artery occlusion. Belumosudil is at least as efficacious in aged, diabetic or female mice, as in normal adult males. There are some clinical trials on going with this molecule in difusse cutaneous systemic sclerosis, chronic plaque psoriasis and chronic Graft-versus-host-disease all sponsored by Kadmon Corporation. Belumosudil formula is:

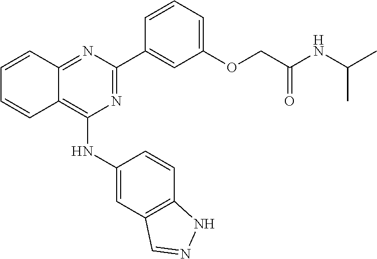 [SMILES: CC(C)NC(=O)COc1cccc(-c2nc(Nc3ccc4[nH]ncc4c3)c3ccccc3n2)c1]

In one example, the inhibitor is Belumosudil or a pharmaceutically acceptable salt, solvate or prodrug thereof.

Belumosudil mesylate (CAS No.: 2109704-99-4) has the structural formula:

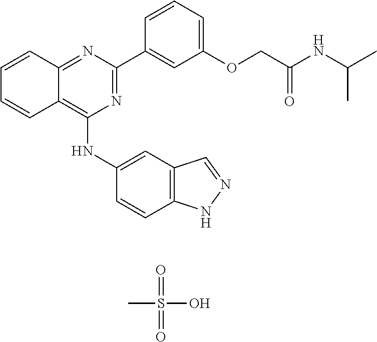

In one example, the inhibitor is Belumosudil or a pharmaceutically acceptable salt, solvate or prodrug thereof. The inhibitor may be Belumosudil mesylate.

AR-13324 (CAS 2309668-15-1) also known as netarsudil is a ROCK and norepinephrine transporter inhibitor that has shown to reduced intraocular pressure in monkey eyes. Netarsudil primarily targets cells in the conventional outflow tract, efficiently decreasing IOP in both human and non-human primate eyes. In addition, netarsudil has been shown to increase outflow facility in non-human primate eyes and to decrease episcleral venous pressure in rabbit eyes. It is currently in clinical trials for the treatment of glaucoma and ocular hypertension. In 2019, Aerie Pharmaceuticals also introduced PG324 (Rocklatan®), an FDA approved fixed-dose combination of Netarsudil (0.02%) and latanoprost (0.005%). AR-13324 has the structural formula

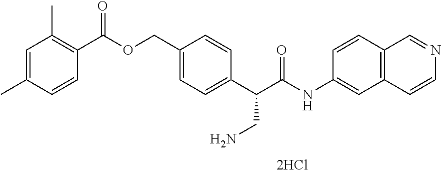 [SMILES: Cc1ccc(C(=O)OCc2ccc([C@@H](CN)C(=O)Nc3ccc4cnccc4c3)cc2)c(C)c1]

In one example, the inhibitor is AR-13324 or a solvate or prodrug thereof, or an alternative pharmaceutically acceptable salt thereof.

Verosudil (also known as AR-11286) is a is a potent, selective Rho-kinase (ROCK) inhibitor with Kis of 2 and 2 nM for ROCK1 and ROCK2, respectively. AR-12286 lowers intraocular pressure (IOP) primarily by increasing aqueous humour outflow through the trabecular meshwork. It is being tested for glaucoma in clinical trials sponsorez by Aerie Pharmaceuticals. Verosudil formula is

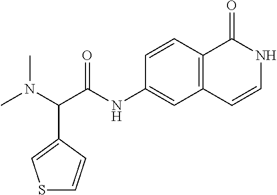 [SMILES: CN(C)C(C(=O)Nc1ccc2c(=O)[nH]ccc2c1)c1ccsc1]

In one example, the inhibitor is Verosudil or a pharmaceutically acceptable salt, solvate or prodrug thereof.

Sovesudil (CAS 1333400-14-8), also known as PHP-201 or AMA0076, is a potent, ATP-competitive, locally acting Rho kinase (ROCK) inhibitor with IC50s of 3.7 and 2.3 nM for ROCK-I and ROCK-II, respectively. Sovesudil hydrochloride is a derivative of sovedusil. Sovesudil lowers intraocular pressure (IOP) without inducing hyperemia. Sovesudil (PHP-201) (1 μM; 60 min) is able to induce altered cellular behavior of human trabecular meshwork (HTM) cells. In vivo experiments showed that Sovesudil (0.1%, 0.3%, and 0.5%) effectively reduces Intraocular Pressure (IOP) in ocular normotensive and acute hypertensive male New Zealand White rabbits without causing distinct hyperemia.

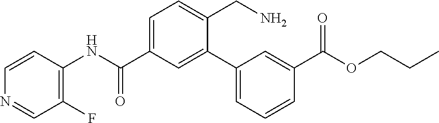 [SMILES: CCCOC(=O)c1cccc(-c2cc(C(=O)Nc3ccncc3F)ccc2CN)c1]

In one example, the inhibitor is Sovesudil or a pharmaceutically acceptable salt, solvate or prodrug thereof.

Aminofurazan (oxadiazole)-azabenzimidazole, is a potent inhibitor of ROCK1 (IC50: 19 nM) that was described in 2007 (Stavenger et al, J Med Chem 2007) with vasodilator effects. The formula is

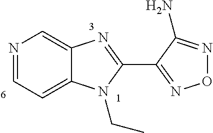 [SMILES: CCn1c(-c2nonc2N)nc2cnccc21]

In one example, the inhibitor is Aminofurazan or a pharmaceutically acceptable salt, solvate or prodrug thereof.

Dihydropyrimidinyl indazole amide were also identified as potent ROCK inhibitors (Goodman et al, J Med Chem, 2007). From this first description other many pyridone derivatives have been created all of them with selective ROCK inhibition potential.

H-1152 (CAS: 451462-58-1) (also known as HMN-1152) is a membrane-permeable and selective ROCK inhibitor, with a Ki value of 1.6 nM, and an IC50 value of 12 nM for ROCK2. There are some derivatives such as H-1152 dihydrochloride or glycyl H-1152 hydrochloride. H-1152 potently inhibits Rho kinase, with a Ki of 1.6 nM, and slightly suppresses PKA, PKC and MLCK, with Kis of 0.63, 9.27, and 10.1 μM, respectively. H-1152 (0.5-10 μM) causes no decreased neuronal survival. H-1152 (1, 5 or 10 μM) also exerts no alterations in the ratios of different neuronal morphologies. Furthermore, H-1152 (10 μM) increases neurite length in both BMP4 and LIF cultures. H-1152 has the structural formula:

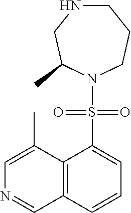 [SMILES: Cc1cncc2cccc(S(=O)(=O)N3CCCNC[C@@H]3C)c12]

In one example, the inhibitor is H-1152 or a pharmaceutically acceptable salt, solvate or prodrug thereof.

XD-4000 is a selective inhibitor of ROCK2. In one example, the inhibitor is XD-4000 or a pharmaceutically acceptable salt, solvate or prodrug thereof.

4-(1-aminoalkyl)-N-(4-pyridyl)cyclohexane-carboxamides were described in 2004 as Rho-kinase inhibitors (Gingrass et al, Biorganic & Medical Chemistry Letters 2004). Several different 4-(1-Aminoalkyl)-N-(4-pyridyl)cyclohexanecarboxamides were examined as inhibitors of Rho kinase and for their ability to stimulate neurite outgrowth on tissue culture treated surface in a semi-quantitative cell-based assay. Enantiomerically enriched (80% ee) compounds were tested directly without further resolution. All compounds inhibited Rho kinase activity at 10 μM by ≥65% relative to vehicle (DMSO) in the presence of 100 μM ATP. Two of the compounds demonstrated similar results for Rho kinase assay. The ability of these compounds to promote neurite outgrowth was demonstrated relative to vehicle. The general formula is:

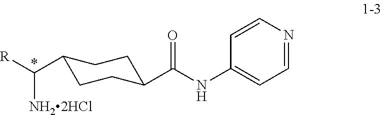

In one example, the inhibitor is a 4-(1-aminoalkyl)-N-(4-pyridyl)cyclohexane-carboxamide or a pharmaceutically acceptable salt, solvate or prodrug thereof.

Quinazoline is an organic compound with the formula C8H6N2. It is an aromatic heterocycle with a bicyclic structure consisting of two fused six-membered aromatic rings, a benzene ring and a pyrimidine ring. Amino acid-derived quinazolines have Rock inhibition potential. Studies have demonstrated that these amino acid derived quinazolinones are mainly pan-Rock (I & II) inhibitors. While selectivity against other kinases could be achieved, selectivity for most of these compounds against PKA was not achieved. This is distinct from Rock inhibitors based on non-amino acid derived quinazolinones, where high selectivity against PKA could be obtained. Quinazolines formula is based on the following:

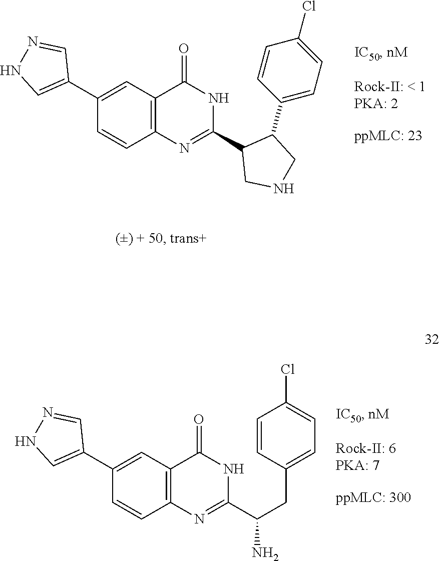

In one example, the inhibitor is a quinazoline or pharmaceutically acceptable salts, solvates or prodrugs thereof.

Ripasudil (CAS 887375-67-9) and derivatives (ripasudil hydrochloride, ripasudil hydrochloride hydrate and ripasudil free base) are derivative of fasudil, that is a rho kinase inhibitor drug (previously known as K-115 used for the treatment of glaucoma and ocular hypertension. Ripasudil has the structural formula

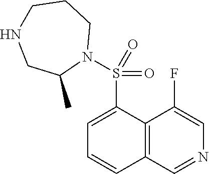 [SMILES: C[C@H]1CNCCCN1S(=O)(=O)c1cccc2cncc(F)c12]

In one example, the inhibitor is Ripasudil or a pharmaceutically acceptable salt, solvate or prodrug thereof.

VAS-012 is a ROCK inhibitor developed by VasGene Therapeutics being investigated in oncology.

Ki-23095 is a ROCK inhibitor being investigated in cardiovascular and renal disease.

BA-2017, BA-215, BA-285, BA-1037, BA-210 are all ROCK inhibitors being developed whose in vivo or in vitro results have not been communicated yet.

Rhosin hydrochloride (CAS: 1173671-63-0) also known as rhostatin, is a is a potent, specific inhibitor of RhoA subfamily Rho GTPases with Kd of ˜0.4 μM. In vitro experiments have shown that the drug inhibits RhoA activity and RhoA-mediated cellular function without affecting Cdc42 or Rac1 signaling activities. By suppressing RhoA or RhoC activity Rhosin can inhibit mammary sphere formation by breast cancer cells, suppress invasion of mammary epithelial cells, and induce neurite outgrowth of PC12 cells in synergy with NGF. Treatment with rhosin abrogates the lung metastasis of B16BL6 and 4T1 cells in vivo in murine animal models.

Rhosin hydrochloride has the structural formula:

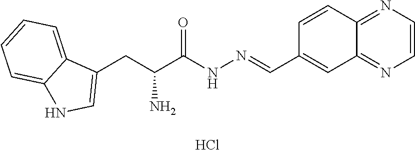 [SMILES: N[C@H](Cc1c[nH]c2ccccc12)C(=O)N/N=C/c1ccc2nccnc2c1 ; Cl]

In one example, the inhibitor is Rhosin or a pharmaceutically acceptable salt, solvate or prodrug thereof. The inhibitor may be Rhosin hydrochloride.

ROCK inhibitor-2 (CAS 1127308-52-4) is a selective dual ROCK1 and ROCK2 inhibitor with IC50s of 17 nM and 2 nM, respectively. The structural formula is

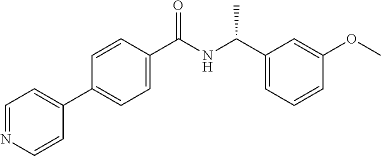 [SMILES: COc1cccc([C@@H](C)NC(=O)c2ccc(-c3ccncc3)cc2)c1]

In one example, the inhibitor is ROCK inhibitor-2 or a pharmaceutically acceptable salt, solvate or prodrug thereof.

ROCK-IN-1 (CAS 934387-35-6) is a potent inhibitor of ROCK, with an IC50 of 1.2 nM for ROCK2. The structural formula is

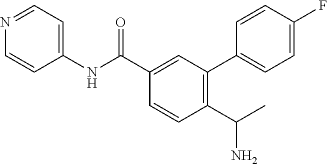 [SMILES: CC(N)c1ccc(C(=O)Nc2ccncc2)cc1-c1ccc(F)cc1]

In one example, the inhibitor is ROCK-IN-1 or a pharmaceutically acceptable salt, solvate or prodrug thereof.

ROCK2-IN-5 (compound 1d) is a hybrid compound containing structural fragments of the Rho kinase inhibitor fasudil and the NRF2 inducers caffeic and ferulic acids. ROCK2-IN-5 has good multitarget profile and good tolerability. ROCK2-IN-5 has the potential for thr research of Amyotrophic lateral sclerosis (ALS) with a SOD1 mutation.

ROCK2-IN-2 (CAS 867017-68-3) is a selective ROCK2 inhibitor extracted from patent US20180093978A1, Compound A-30, has an IC50 of <1 μM. The structural formula is

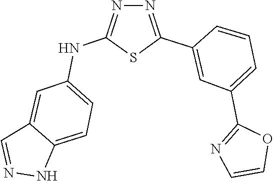 [SMILES: c1cc(-c2ncco2)cc(-c2nnc(Nc3ccc4[nH]ncc4c3)s2)c1]

In one example, the inhibitor is ROCK2-IN-2 or a pharmaceutically acceptable salt, solvate or prodrug thereof.

Hydroxyfasudil (CAS No.: 105628-72-6) is a ROCK inhibitor, with IC50s of 0.73 and 0.72 μM for ROCK1 and ROCK2, respectively and has the structural formula:

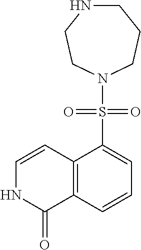 [SMILES: O=c1[nH]ccc2c(S(=O)(=O)N3CCCNCC3)cccc12]

In one example, the inhibitor is Hydroxyfasudil or a pharmaceutically acceptable salt, solvate or prodrug thereof.

Hydroxyfasudil hydrochloride (CAS No.: 155558-32-0) has the structural formula:

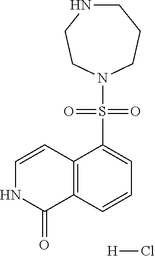

In one example, the inhibitor is Hydroxyfasudil or a pharmaceutically acceptable salt, solvate or prodrug thereof. The inhibitor may be Hydroxyfasudil dichloride.

Ripasudil (K-115) (CAS No.: 887375-67-9) is a specific inhibitor of ROCK, with IC50s of 19 and 51 nM for ROCK2 and ROCK1, respectively and the hydrochloride salt has the structural formula:

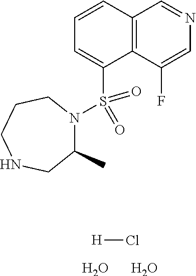

In one example, the inhibitor is Ripasudil hydrochloride or a solvate or prodrug thereof, or an alternative pharmaceutically acceptable salt thereof.

Ripasudil free base (CAS No.: 223645-67-8) has the structural formula:

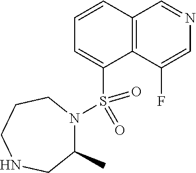 [SMILES: C[C@H]1CNCCCN1S(=O)(=O)c1cccc2cncc(F)c12]

In one example, the inhibitor is Ripasudil free base or a pharmaceutically acceptable salt, solvate or prodrug thereof.

The inhibitor Chroman 1 (CAS No.: 1273579-40-0), as well as its salts, solvates and salts of the solvates is a highly potent and selective ROCK inhibitor. Chroman 1 is more potent against ROCK2 (IC50=1 pM) than ROCK1 (IC50=52 pM). Chroman 1 also has inhibitory activity against MRCK, with an IC50 of 150 nM. Chroman 1 has the structural formula:

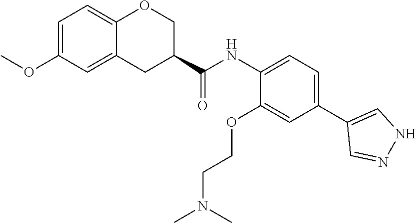 [SMILES: COc1ccc2c(c1)C[C@H](C(=O)Nc1ccc(-c3cn[nH]c3)cc1OCCN(C)C)CO2]

In one example, the inhibitor is Chroman 1 or a pharmaceutically acceptable salt, solvate or prodrug thereof.

Chroman 1 dihydrochloride has the structural formula:

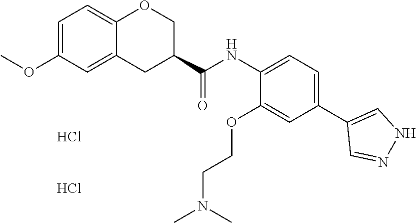 [SMILES: COc1ccc2c(c1)C[C@H](C(=O)Nc1ccc(-c3cn[nH]c3)cc1OCCN(C)C)CO2]

In one example, the inhibitor is Chroman 1 dihydrochloride a pharmaceutically acceptable salt, solvate or prodrug thereof.

The inhibitor Cotosudil (CAS No. 1258833-31-6) is a ROCK kinase inhibitor, which can be used for glaucoma or ocular hypertension research. Cotosudil Chroman 1 has the structural formula:

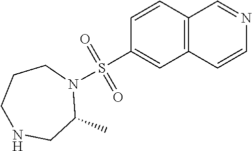 [SMILES: C[C@@H]1CNCCCN1S(=O)(=O)c1ccc2cnccc2c1]

In one example, the inhibitor is Cotosudil 1 or a pharmaceutically acceptable salt, solvate or prodrug thereof.

The inhibitor SAR407899 (CAS No.: 923359-38-0), as well as its salts, solvates and salts of the solvates, is a selective, potent and ATP-competitive ROCK inhibitor, with an IC50 of 135 nM for ROCK-2, and Kis of 36 nM and 41 nM for human and rat ROCK-2, respectively. In vivo, SAR407899 inhibits ROCK-mediated phosphorylation of MYPTT696 in thoracic aorta of spontaneously hypertensive rats and efficiently reduces pressor responses to vasoconstrictor agents in rats. SAR407899 has the structural formula:

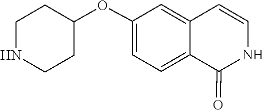 [SMILES: O=c1[nH]ccc2cc(OC3CCNCC3)ccc12]

In one example, the inhibitor is SAR407899 or a pharmaceutically acceptable salt, solvate or prodrug thereof.

SAR407899 hydrochloride has the structural formula:

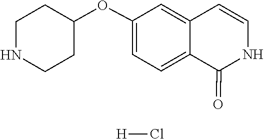 [SMILES: O=c1[nH]ccc2cc(OC3CCNCC3)ccc12 ; Cl]

In one example, the inhibitor is SAR407899 or a pharmaceutically acceptable salt, solvate or prodrug thereof. The inhibitor may be SAR407899 hydrochloride.

Sovesudil (PHP-201) (CAS No.: 1333400-14-8) as well as its salts, solvates and salts of the solvates, is a potent, ATP-competitive, locally acting Rho kinase (ROCK) inhibitor with IC50s of 3.7 and 2.3 nM for ROCK-I and ROCK-II, respectively. Sovesudil lowers intraocular pressure (IOP) without inducing hyperemia and has the structural formula:

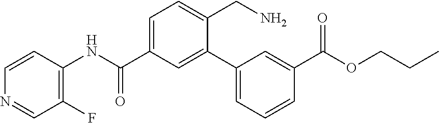 [SMILES: CCCOC(=O)c1cccc(-c2cc(C(=O)Nc3ccncc3F)ccc2CN)c1]

In one example, the inhibitor is Sovesudil or a pharmaceutically acceptable salt, solvate or prodrug thereof.

Sovesudil hydrochloride has the structural formula:

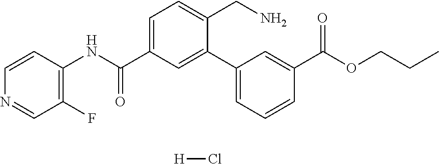 [SMILES: CCCOC(=O)c1cccc(-c2cc(C(=O)Nc3ccncc3F)ccc2CN)c1 ; Cl]

In one example, the inhibitor is Sovesudil hydrochloride or a solvate or prodrug thereof, or an pharmaceutically acceptable salt, thereof.

RKI 1447 (CAS No. 1342278-01-6) as well as its salts, solvates and salts of the solvates, is a potent inhibitor of the Rho-associated ROCK kinases with anti-invasive and antitumor activities in breast cancer (IC50 values 14.5 and 6.2 nM for ROCK 1 and 2 respectively). RKI 1447 is a Type 1 inhibitor that binds both the hinge region and the DFG motif of the ROCK ATP binding site. RKI 1447 has the structural formula:

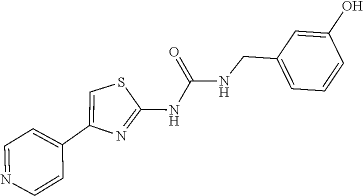 [SMILES: O=C(NCc1cccc(O)c1)Nc1nc(-c2ccncc2)cs1]

In one example, the inhibitor is RKI 1447 or a pharmaceutically acceptable salt, solvate or prodrug thereof.

RKI 1447 dihydrochloride (CAS No. 1782109-09-4) has the structural formula:

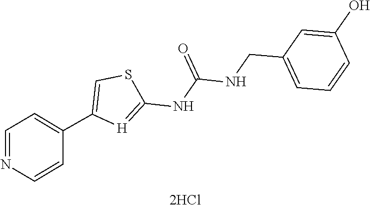

In one example, the inhibitor is RKI 1447 or a pharmaceutically acceptable salt, solvate or prodrug thereof. The inhibitor may be RKI 1447 hydrochloride.

H-1152 (CAS No.: 451462-58-1) as well as its salts, solvates and salts of the solvates, is a membrane-permeable and selective ROCK inhibitor, with a Ki value of 1.6 nM, and an IC50 value of 12 nM for ROCK2. H-1152 also shows less inhibitory activities against CaMKII, PKG, AuroraA, PKA, Src, PKC, MLCK, Abl, EGFR, MKK4, GSK3a, AMPK, and P38α. H-1152 has the structural formula:

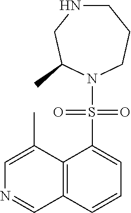 [SMILES: Cc1cncc2cccc(S(=O)(=O)N3CCCNC[C@@H]3C)c12]

In one example, the inhibitor is H-1152 or a pharmaceutically acceptable salt, solvate or prodrug thereof.

H-1152 dihydrochloride (CAS No.: 871543-07-6) has the structural formula:

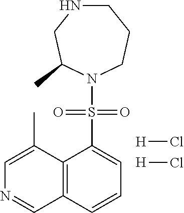

In one example, the inhibitor is H-1152 dichloride or a solvate or prodrug thereof, or an alternative pharmaceutically acceptable salt thereof.

GSK269962A (GSK 269962) (CAS No.: 850664-21-0), as well as its salts, solvates and salts of the solvates, is a potent ROCK inhibitor with IC50s of 1.6 and 4 nM for recombinant human ROCK1 and ROCK2 respectively. GSK269962A has anti-inflammatory and vasodilatory activities. GSK269962A is a potent antihypertensive agent that induces a dose-dependent reduction in blood pressure in spontaneously hypertensive rat. The structural formula is:

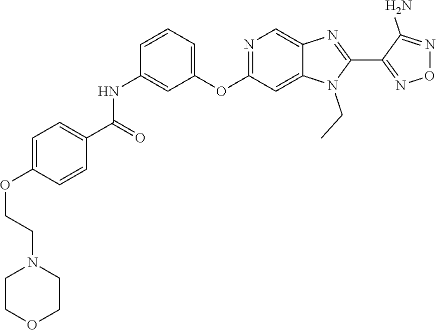 [SMILES: CCn1c(-c2nonc2N)nc2cnc(Oc3cccc(NC(=O)c4ccc(OCCN5CCOCC5)cc4)c3)cc21]

In one example, the inhibitor is GSK269962A or a pharmaceutically acceptable salt, solvate or prodrug thereof.

GSK269962A hydrochloride (GSK 269962 hydrochloride) (CAS No.: 2095432-71-4) has the structural formula:

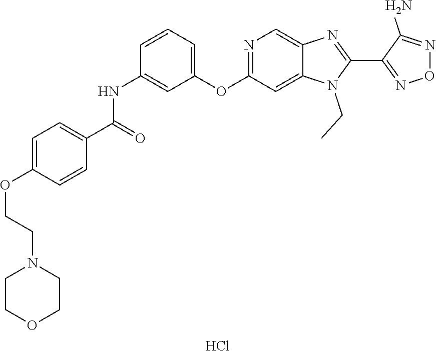 [SMILES: CCn1c(-c2nonc2N)nc2cnc(Oc3cccc(NC(=O)c4ccc(OCCN5CCOCC5)cc4)c3)cc21 ; Cl]

In one example, the inhibitor is GSK269962A or a pharmaceutically acceptable salt, solvate or prodrug thereof. The inhibitor may be GSK269962A hydrochloride

Rho-Kinase-IN-1 (CAS No.: 1035094-83-7) is a Rho kinase (ROCK) inhibitor (Ki values of 30.5 and 3.9 nM for ROCK1 and ROCK2, respectively) extracted from US20090325960A1, compound 1.008. Rho-Kinase-IN-1 can be useful for treating diseases or conditions associated with excessive cell proliferation, remodeling, edema and inflammation and has the structural formula:

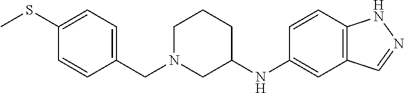 [SMILES: CSc1ccc(CN2CCCC(Nc3ccc4[nH]ncc4c3)C2)cc1]

In one example, the inhibitor is Rho-Kinase-IN-1 or a pharmaceutically acceptable salt, solvate or prodrug thereof.

SB-772077B dihydrochloride (CAS No.: 607373-46-6) is an aminofurazan-based Rho kinase (ROCK) inhibitor with IC50s of 5.6 nM and 6 nM toward ROCK1 and ROCK2, respectively and has the structural formula:

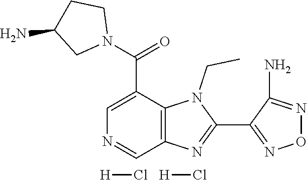

In one example, the inhibitor is SB-772077B or a pharmaceutically acceptable solvate or prodrug thereof. The inhibitor may be SB-772077B dihydrochloride.

HSD1590 (CAS No.: 2379279-96-4) is potent ROCK inhibitor, with IC50s of 1.22 and 0.51 nM for ROCK1 and ROCK2, respectively. HSD1590 exhibits single digit nanomolar binding to ROCK (Kds<2 nM). In vitro studies shows that HSD1590 exhibits an impressive attenuation in migration. HSD1590 has the structural formula:

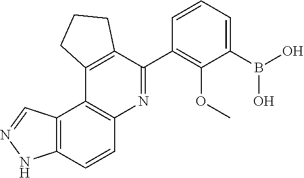 [SMILES: COc1c(B(O)O)cccc1-c1nc2ccc3[nH]ncc3c2c2c1CCC2]

In one example, the inhibitor is HSD1590 or a pharmaceutically acceptable salt, solvate or prodrug thereof.

BDP5290 (CAS No.: 1817698-21-7) is a potent inhibitor of both ROCK and MRCK with IC50s of 5 nM, 50 nM, 10 nM and 100 nM for ROCK1, ROCK2, MRCKα and MRCKβ, respectively. In vitro studies shows that BDP5290 completely inhibits myosin II light chain (MLC) phosphorylation induced by MRCKβ. BDP5290 has the structural formula:

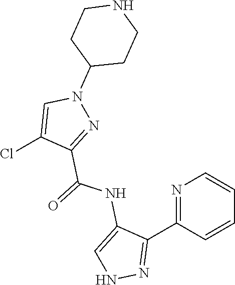 [SMILES: O=C(Nc1c[nH]nc1-c1ccccn1)c1nn(C2CCNCC2)cc1Cl]

In one example, the inhibitor is BDP5290 or a pharmaceutically acceptable salt, solvate or prodrug thereof.

ZINC00881524 (CAS No.: 557782-81-7) is a Rho-associated kinase (ROCK) inhibitor. In vitro, it decreases levels of ROCK1 in T47D and CAMA-1 breast cancer cells. ZINC00881524 decreases proliferation of T47D and CAMA-1 cells when used in combination with CNN1 knockdown. ZINC00881524 has the structural formula:

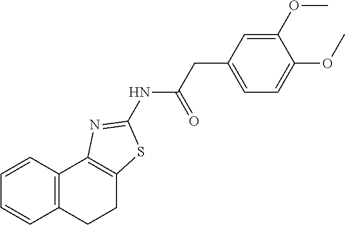 [SMILES: COc1ccc(CC(=O)Nc2nc3c(s2)CCc2ccccc2-3)cc1OC]

In one example, the inhibitor is ZINC00881524 or a pharmaceutically acceptable salt, solvate or prodrug thereof.

Verosudil (AR-12286) (CAS No.: 1414854-42-4) is a potent, selective Rho-kinase (ROCK) inhibitor with Kis of 2 and 2 nM for ROCK1 and ROCK2, respectively. AR-12286 lowers intraocular pressure (IOP) primarily by increasing aqueous humour outflow through the trabecular meshwork. Verosudil has the structural formula:

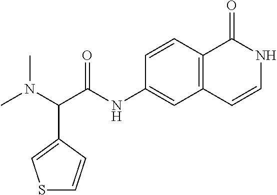 [SMILES: CN(C)C(C(=O)Nc1ccc2c(=O)[nH]ccc2c1)c1ccsc1]

In one example, the inhibitor is Verosudil or a pharmaceutically acceptable salt, solvate or prodrug thereof.

LX7101 (CAS No.: 1192189-69-7) is a potent inhibitor of LIMK and ROCK2 with IC50 values of 24, 1.6 and 10 nM for LIMK1, LIMK2 and ROCK2, respectively; also inhibits PKA with an IC50 less than 1 nM. LX7101 has the structural formula:

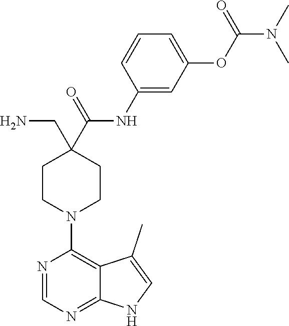 [SMILES: Cc1c[nH]c2ncnc(N3CCC(CN)(C(=O)Nc4cccc(OC(=O)N(C)C)c4)CC3)c12]

In one example, the inhibitor is LX7101 or a pharmaceutically acceptable salt, solvate or prodrug thereof.

GSK-25 (CAS No.: 874119-56-9) is a potent, selective and orally bioavailable ROCK1 inhibitor (IC50=7 nM). GSK-25 maintains good selectivity against RSK1 and p70S6K (RSK1: IC50=398 nM, p70S6K: IC50=1 μM). GSK-25 inhibits P450 profile (IC50s of 2.5, 5.2, 2.5 μM for CYP2C9, CYP2D6, CYP3A4, respectively). GSK-25 is profiled in a spontaneously hypertensive rat (SHR) model of hypertension that induces drop in blood pressure. GSK-25 has the structural formula:

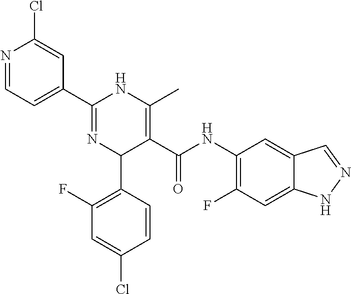 [SMILES: CC1=C(C(=O)Nc2cc3cn[nH]c3cc2F)C(c2ccc(Cl)cc2F)N=C(c2ccnc(Cl)c2)N1]

In one example, the inhibitor is GSK-25 or a pharmaceutically acceptable salt, solvate or prodrug thereof.

GSK180736A (CAS No.: 817194-38-0) is potent Rho-associated coiled-coil kinase 1 (ROCK1) inhibitor with an IC50 of 100 nM. GSK180736A is also a selective and ATP-competitive G protein-coupled receptor kinase 2 (GRK2) inhibitor with an IC50 of 0.77 μM. GSK180736A is a compound structurally similar to paroxetine that is developed as a ROCK inhibitor, is shown to be an even more potent and selective inhibitor of GRK2 with an IC50 of 0.77 μM and more than 100-fold selectivity over other GRKs. ROCK1 is a potential therapeutic target in the treatment of cardiovascular diseases such as hypertension and has the structural formula:

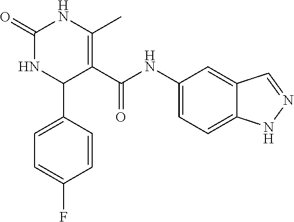 [SMILES: CC1=C(C(=O)Nc2ccc3[nH]ncc3c2)C(c2ccc(F)cc2)NC(=O)N1]

In one example, the inhibitor is GSK180736A or a pharmaceutically acceptable salt, solvate or prodrug thereof.

CRT0066854 (CAS No.: 1438881-19-6), as well as its salts, solvates and salts of the solvates, is a potent and selective atypical PKC isoenzymes inhibitor. CRT0066854 is against full-length (FL) PKCι, PKCζ, and ROCK-II kinases with IC50 values of 132 nM, 639 nM, and 620 nM, respectively. CRT0066854 has the structural formula:

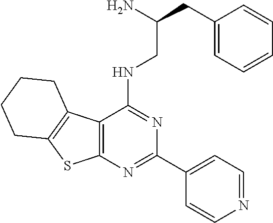 [SMILES: N[C@H](CNc1nc(-c2ccncc2)nc2sc3c(c12)CCCC3)Cc1ccccc1]

In one example, the inhibitor is CRT0066854 or a pharmaceutically acceptable salt, solvate or prodrug thereof.

CRT0066854 hydrochloride (CAS No.: 2250019-91-9) has the structural formula:

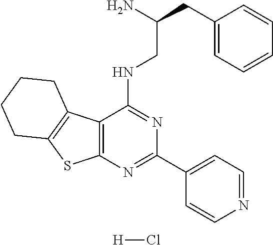 [SMILES: N[C@H](CNc1nc(-c2ccncc2)nc2sc3c(c12)CCCC3)Cc1ccccc1 ; Cl]

In one example, the inhibitor is CRT0066854 or a pharmaceutically acceptable salt, solvate or prodrug thereof. The inhibitor may be CRT0066854 hydrochloride.

In one example, the inhibitor is selected from the group consisting of C3 exoenzyme, C3 Trans based, Rhosin, Rhosin hydrochloride, CCG-1423, CCG-203971, YS-49 monohydrate, YS-49, Cerivastatin sodium, Cerivastatin, Z62954982, Y16, MLS-573151, HA-100 hydrochloride, HA-100, HL07, DDO-5701, DDO-5713, DDO-5714, DDO-5715, DDO-5716, ML-7, MLCK18, and CT-04. These inhibitors inhibit the RhoA/ROCK pathway at different stages, but they do not inhibit ROCK directly.

The RhoA/ROCK2 signalling pathway is well known for regulating actin cytoskeleton organization and cellular dynamics in several cell types. RhoA acts through two molecular conformations: it is inactive when bound to a guanosine diphosphate (GDP) and active when bound to GTP.ROCK, the Rho downstream effector molecule, is a serine/threonine kinase protein that regulates actin filament remodelling by phosphorylating numerous downstream target proteins, including the myosin binding subunit of myosin light chain 2 (MLC2) phosphatase. So, inactivation of RhoA/ROCK pathway can be driven by targeting different proteins of the signalling pathway.

In one example, the inhibitor inhibits the RhoA/ROCK pathway by ribosylating RhoA proteins. In other words, the inhibitor may be an ADP ribosyl transferase.

Ribosylating the RhoA proteins involved in the RhoA/ROCK pathway renders RhoA inactive, resulting in a block of the whole pathway (FIG. 3A) which inhibits cytoskeletal rearrangement.

In one example, the inhibitor is C3 exoenzyme. C3 exoenzyme inhibits the RhoA/ROCK pathway by ribosylating RhoA. C3 exoenzyme is an ADP ribosyl transferase.

“C3 exoenzyme” or “C3 transferase” (UniProtKB-Q7M0L1 (Q7M0L1_CLOBO) is an enzyme from Clostridium botulinum that blocks RhoA by locking RhoA in an inactive state and keeping it bound to a RhoA dissociation inhibitor. C3 exoenzyme is an enzyme from Clostridium botulinum, C3 transferase, and selectively blocks Rho A, B, and C function by ADP ribosylation on asparagine 41, without affecting Rac or Cdc42 (FIG. 3A).

Rhosin (CAS No.: 1173671-63-0) is a potent, specific RhoA subfamily Rho GTPases inhibitor, which specifically binds to RhoA to inhibit RhoA-GEF interaction with a Kd of ˜0.4 μM, and does not interact with Cdc42 or Rac1, nor the GEF, LARG. Rhosin induces cell apoptosis. Rhosin promotes stress resiliency through enhancing D1-MSN plasticity and reducing hyperexcitability. The compound of Rhosin is

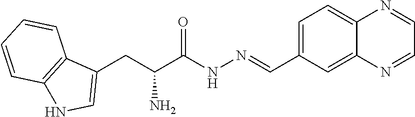 [SMILES: N[C@H](Cc1c[nH]c2ccccc12)C(=O)N/N=C/c1ccc2nccnc2c1]

In one example, the inhibitor is Rhosin or a pharmaceutically acceptable salt, solvate or prodrug thereof.

CCG-1423 (CAS No.: 285986-88-1) is a novel inhibitor of RhoA/C-mediated gene transcription that is capable of inhibiting invasion of PC-3 prostate cancer cells in a Matrigel model of metastasis. IC50 value: 1.5 μM Target: Rho signaling inhibitor in vitro: CCG-1423 selectively inhibited spontaneous PC-3 prostate cancer cell invasion through a Matrigel matrix, but not the Gai-dependent LPA-stimulated SKOV-3 ovarian cancer cell invasion, in vitro. At 100 μM, nearly complete inhibition of invasion was achieved with a lesser degree of toxicity than that induced by CCG-1423 at 10 μM. SRF binds to this site in vivo and the SRF inhibitor CCG-1423 completely blocks STARS proximal reporter activity in H9c2 cells. pharmacological MKL-inhibition with CCG-1423 significantly inhibited CCN1 promoter activity as well as mRNA and protein expression. in vivo: Pharmacological SRF inhibition by CCG-1423 reduced nuclear MKL1 and improved glucose uptake and tolerance in insulin-resistant mice in vivo. The compound of CCG-1423 is

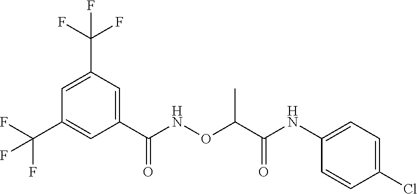 [SMILES: CC(ONC(=O)c1cc(C(F)(F)F)cc(C(F)(F)F)c1)C(=O)Nc1ccc(Cl)cc1]

In one example, the inhibitor is CCG-1423 or a pharmaceutically acceptable salt, solvate or prodrug thereof.

CCG-203971 (CAS No 1443437-74-8) is a second-generation Rho/MRTF/SRF pathway inhibitor. CCG-203971 potently targets RhoA/C-activated SRE-luciferase (IC50=6.4 μM). CCG-203971 inhibits PC-3 cell migration with an IC50 of 4.2 μM. Potential anti-metastasis Agent. In vitro, CCG-203971, a second-generation Ras homolog gene family, member A (RhoA)/myocardin-related transcription factor A (MRTF-A)/serum response factor (SRF) pathway inhibitor, represses both matrix-stiffness and transforming growth factor beta-mediated fibrogenesis as determined by protein and gene expression in a dose-dependent manner. CCG-203971 significantly represses TGF-β-induced MKL1 expression at 25 μM concentration. The compound of CCG-203971 is

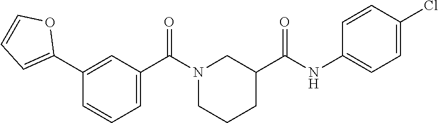 [SMILES: O=C(Nc1ccc(Cl)cc1)C1CCCN(C(=O)c2cccc(-c3ccco3)c2)C1]

In one example, the inhibitor is CCG-203971 or a pharmaceutically acceptable salt, solvate or prodrug thereof.

YS-49 (CAS No 132836-42-1) is a PI3K/Akt (a downstream target of RhoA) activator, to reduce RhoA/PTEN activation in the 3-methylcholanthrene-treated cells. YS-49 inhibits angiotensin II (Ang II)-stimulated proliferation of VSMCs via induction of heme oxygenase (HO)-1. YS-49 is also an isoquinoline compound alkaloid, has a strong positive inotropic action through activation of cardiac β-adrenoceptors.

In vitro, YS-49 (1-10 μM; 18 hours; RAVSMC and RAW 264.7 cells) concentration-dependently inhibits the accumulation of nitrite in both RAVSMC and RAW 264.7 exposed to lipopolysaccharide (LPS) plus INF-y, with IC50 values of 22 μM and 30 μM, respectively YS-49 (10-100 μM; 18 hours; RAVSMC and RAW 264.7 cells) suppresses iNOS gene expression induced by LPS and/or cytokines in RAVSMC and RAW 264.7 cells at the transcriptional level. The compound of YS-49 is

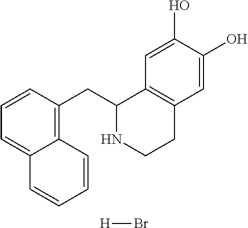

In one example, the inhibitor is YS-49 or a pharmaceutically acceptable salt, solvate or prodrug thereof.

Cerivastatin (CAS No 145599-86-6) is a synthetic lipid-lowering agent and a highly potent, well-tolerated and orally active HMG-COA reductase inhibitor, with a Ki of 1.3 nM/L. Cerivastatin reduces low-density lipoprotein cholesterol levels. Cerivastatin also inhibits proliferation and invasiveness of MDA-MB-231 cells, mainly by RhoA inhibition, and has anti-cancer effect. In vitro, Cerivastatin induces decrease in cell proliferationm increase in the level of p21, inhibits invasion of cells, inactivates NFkB. The compound of Cerivastatin is

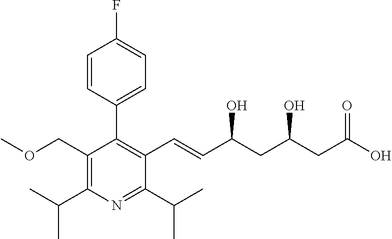 [SMILES: COCc1c(C(C)C)nc(C(C)C)c(/C=C/[C@@H](O)C[C@@H](O)CC(=O)O)c1-c1ccc(F)cc1]

In one example, the inhibitor is Cerivastatin or a pharmaceutically acceptable salt, solvate or prodrug thereof.

Z62954982 (CAS No 1090893-12-1) is a potent, selective and cell-permeable Rac1 (IC50=12 μM) inhibitor that is 4 times more effective than NSC23766 (HY-15723A) (IC50=50 μM). Z62954982 disrupts the Rac1/Tiam 1 complex and decreases cytoplasmic levels of active Rac1 (GTP-bound Rac1), without affecting the activity of other Rho GTPases (such as Cdc42 or RhoA. The compound of Z62954982 is

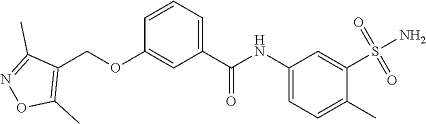 [SMILES: Cc1ccc(NC(=O)c2cccc(OCc3c(C)noc3C)c2)cc1S(N)(=O)=O]

In one example, the inhibitor is Z62954982 or a pharmaceutically acceptable salt, solvate or prodrug thereof.

Y16 (CAS No 429653-73-6) is a specific inhibitor of Leukemia-associated Rho guanine nucleotide exchange factor (LARG) with a Kd value of 76 nM. Y16 is active in blocking the interaction of LARG and related G-protein-coupled Rho GEFs with RhoA. Y16 shows no detectable effect on other diffuse B-cell lymphoma (Dbl) family Rho GEFs, Rho effectors, or a RhoGAP. In vitro, Y16 (10-30 μM; 24 hours; NIH 3T3 cells) could inhibit RhoA-GTP formation induced by serum dose dependently and is specific for RhoA. Y16 (10-30 μM; 24 hours; NIH 3T3 cells) efficiently inhibits serum or SDF-1α-induced phospho-MLC and phospho-FAK formation, which are downstream of RhoA. The compound of Y16 is

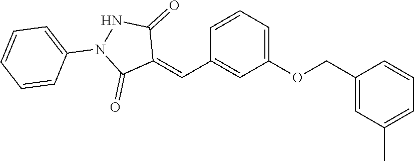 [SMILES: Cc1cccc(COc2cccc(/C=C3\C(=O)NN(c4ccccc4)C3=O)c2)c1]

In one example, the inhibitor is Y16 or a pharmaceutically acceptable salt, solvate or prodrug thereof.

MLS-573151 (CAS No 10179-57-4) is a selective GTPase Cdc42 inhibitor with an EC50 of 2 μM. MLS-573151 is inactive against other GTPases family members, such as Rab2, Rab7, H-Ras, Rac1, Rac 2 and RhoA wild-type. MLS-573151 acts by blocking the binding of GTP to Cdc42. In vitro, The fluorescence intensities of phagocytosed beads or bacteria in hemocytes, taken as a measure of phagocytosis efficiency, were markedly reduced in granulocytes treated with MLS-573151 (50 μM; for 15 min) compared to that in the control group. MLS-573151 could effectively inhibit the phagocytic ability of granulocytes. The compound of MLS-573151 is

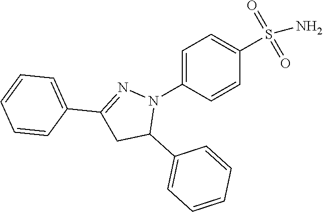 [SMILES: NS(=O)(=O)c1ccc(N2N=C(c3ccccc3)CC2c2ccccc2)cc1]

In one example, the inhibitor is MLS-573151 or a pharmaceutically acceptable salt, solvate or prodrug thereof.

HA-100 (CAS No: 84468-24-6) is a potent protein kinase inhibitor, with IC50s of 4 μM, 8 μM, 12 μM and 240 μM for cGMP-dependent protein kinase (PKG), cAMP-dependent protein kinase (PKA), protein kinase C (PKC) and MLC-kinase, respectively. HA-100 also used as a ROCK inhibitor. In vitro, HA-100 inhibits MLC-kinase and PKC competitively with respect to ATP, with Kis of 61 and 6.5 μM, respectively. The compound of HA-100 is

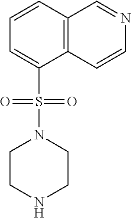 [SMILES: O=S(=O)(c1cccc2cnccc12)N1CCNCC1]

In one example, the inhibitor is HA-100 or a pharmaceutically acceptable salt, solvate or prodrug thereof.

HL-07 is an inhibitor of the activation of RhoA preferently over GNP. HL07 inhibited phenylephrine (PE)-induced contraction in rat aorta and blocked RhoA activation stimulated by PE in human cerebrovascular smooth muscle cells. The compound of HL-07 is

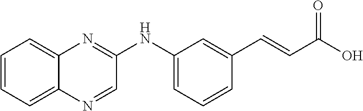 [SMILES: O=C(O)/C=C/c1cccc(Nc2cnc3ccccc3n2)c1]

In one example, the inhibitor is HL-07 or a pharmaceutically acceptable salt, solvate or prodrug thereof.

DDO-5701 or Proglumide (CAS No.: 6620-60-6) (approved drug) is a nonpeptide and orally active cholecystokinin (CCK)-A/B receptors antagonist. Proglumide selective blocks CCK's effects in the central nervous system. Proglumide has the ability to inhibit gastric secretion and to protect the gastroduodenal mucosa. Proglumide also has antiepileptic and antioxidant activities. The compound of DDO-5701 is

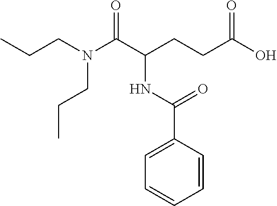 [SMILES: CCCN(CCC)C(=O)C(CCC(=O)O)NC(=O)c1ccccc1]

In one example, the inhibitor is DDO-5701 or a pharmaceutically acceptable salt, solvate or prodrug thereof.

DDO-5713, DDO-5714, DDO-5715, DDO-5716 are DDO-5701 derivates by structural modifications considering binding activity. They are small molecule inhibitor of RhoA and exhibit higher RhoA inhibition activities than DDO-5701. Among these 4 inhibitors, DDO-5716 has shown potential use for treating breast cancer. More in detail, DDO-5716 can effectively reverse the functions of breast cancer cells regulated by RhoA.

ML-07 hydrochloride (CAS No: 110448-33-4) is a naphthalene sulphonamide derivative, potently inhibits MLCK (IC50=300 nM). ML-7 hydrochloride also inhibits YAP/TAZ. Displays more potent inhibition than ML 9 hydrochloride. Displays reversible, ATP-competitive inhibition of Ca2+-calmodulin-dependent and -independent smooth muscle MLCKs. Inhibits proplatelet formation and stabilization. The compound of ML-07 is

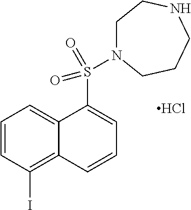 [SMILES: O=S(=O)(c1cccc2c(I)cccc12)N1CCCNCC1 ; Cl]

In one example, the inhibitor is ML-07 or a pharmaceutically acceptable salt, solvate or prodrug thereof. The inhibitor may be ML-07 hydrochloride.

MLCK18: (CAS No.: 224579-74-2): MLCK inhibitor peptide 18 is a myosin light chain kinase (MLCK) inhibitor with an IC50 of 50 nM and inhibits CaM kinase II at 4000-fold selectivity without inhibiting PKA.

The amino acid sequence of MLCK18 is RKKYKYRRK (SEQ ID NO:1).

### Uses

The RhoA/ROCK pathway inhibitors described herein can be used for treating a disease or disorder associated with muscle fibrosis.

The RhoA/ROCK pathway inhibitors described herein can be used in the manufacture of a medicament for the treatment of a disease or disorder associated with muscle fibrosis.

Details with regards to the inhibitors, treatment, and the diseases or disorders are described elsewhere herein and equally apply here.

A pharmaceutical composition is also provided herein, wherein the composition comprises at least one RhoA/ROCK pathway inhibitor described herein and a pharmaceutically acceptable excipient, adjuvant, diluent and/or carrier. Compositions may routinely contain pharmaceutically acceptable concentrations of salt, buffering agents, preservatives, compatible carriers, supplementary immune potentiating agents such as adjuvants and cytokines and optionally other therapeutic agents or compounds.

In one embodiment the pharmaceutical composition comprises only one RhoA/ROCK pathway inhibitor as active agent. In a preferred embodiment the only RhoA/ROCK pathway inhibitor in the composition is Fasudil or C3 exoenzyme.

In one embodiment a pharmaceutical composition is provided comprising a RhoA/ROCK pathway inhibitor as active agent for treating a disease or disorder associated with muscle fibrosis, in particular DMD or Becker muscular dystrophy.

Details with regards to the inhibitors, treatment, and the diseases or disorders are described elsewhere herein and equally apply here.

An inhibitor of the invention can be for use or administration alone or in combination with at least one or more other compounds. Administration “in combination with” at least one or more other compounds includes simultaneous (concurrent) and consecutive administration in any order. “Combined use” and “combination” in the context of the invention also includes a pharmaceutical product comprising both the inhibitor and at least one or more other compounds, as discrete separate dosage forms, in separate containers or e. g. in blisters containing both types of drugs in discrete solid dosage units, e.g. in a form in which the dosage units which have to be taken together or which have to be taken within one day are grouped together in a manner which is convenient for the patient. Said pharmaceutical product itself or as a part of a kit may contain instructions for the simultaneous, sequential or separate administration of the discrete separate dosage units, to a subject in need thereof.

### Outcome Measures and Dosages

Outcome measures for an individual with a MD vary according to the progression of the disease and the age of a patient.

In certain embodiments, administering the inventive inhibitor affects clinical outcomes of disease. Some commonly used outcome measures are described in the following section.

Generally, these assessments are repeated over time and during the natural history of MD, performance will deteriorate. In general, patients treated according to the invention will deteriorate at a slower rate than patients treated in accordance with the standard of care. Some patients may improve. In some embodiments, treated patients will have stable scores on one or more of these measures for 3, 6, 9 or 12 months or longer when treated according to the invention. In certain embodiments, patients treated according to the invention will show a rate of deterioration on one or more of these measures that is at least 5%, 10%, 20%, 30%, 40% or a greater percentage as compared to patients just treated with standard of care at 6 months, 12 months, 18 months, 24 months or even longer periods of time.

The 6-minute walking test measures the distance the subject can walk in six minutes. Reduced distance indicates reduced function and increased distance, increased function. This test is one of the most used tests and has been used as the basis of regulatory drug approvals.

The North Star ambulatory assessment scale (NSAA) is a 17-item rating scale that is used to measure functional motor abilities in ambulant children with MD. Each item is scored 0, 1 or 2, with a score of 2 assigned for “normal” performance, with the activity performed as instructed, a 1 assigned if the subject did not perform as instructed but achieved the goal independent of physical assistance from another and 0 is assigned if the subject is unable to achieve the goal independently. The NSAA is also very commonly used in ambulatory patients.

The Performance of upper limb (PUL) assesses upper limb performance and, thus, it can be applied to non-ambulatory patients. There are two iterations of PUL, 1.2 and 2.0 that are commonly used, each assesses 3 “dimensions” of upper limb performance: high/shoulder-level, mid/elbow level, and distal/wrist and finger level. PUL 1.2 assesses 22 items with a maximum score of 72 (high=4 items for 16 possible points; mid=9 items for 34 possible points; and distal=8 items for 24 possible points). PUL 2.0 substantially overlaps with PUL 1.2, though several assessments were deleted as redundant and the scoring range was reduced to 0, 1, or 2 for many assessments, similar to the NSAA scoring. Thus, the total possible score for PUL 2.0 is 42 (12 for the high/shoulder level, 17 for the mid/elbow level, and 13 for the distal/wrist/hand level). A higher score indicates greater function on both versions.

Other simple motor assessments may also be used. For example, the time taken to climb four or the time taken to rise from the floor. Shorter times indicate better function. Measures of breathing performance, such as percent predicted forced vital capacity, percent predicted peak expiratory flow or slow vital capacity may be assessed by spirometry. Change in the grip strength of the hands may be assessed by hand-held myometry/dynamometry.

Patients with MD often suffer from cardiomyopathy and the inventive methods are contemplated to improve cardiac function. Cardiomyopathy is the measurable deterioration of the myocardium's ability to contract, leading to heart failure. The disease progresses over time with variable onset of arrhythmias and ventricle dysfunction. Electrocardiographic abnormalities can be found early in the disease and progress with age. Development of cardiomyopathy is characterized by initial diastolic dysfunction followed by eccentric hypertrophy. Cardiomyopathic symptoms may be measured, for example, using echocardiographic evaluation, cardia magnetic resonance imagining (MRI), cardiac MRI with late gadolinium enhancement. In particular, the left ventricular size, thickness, volumes, ejection fraction, and scar/inflammatory burden of the myocardium, as well as strain may be evaluated. Strain, change in left ventricular dimension and volume, and scar burden are key measures.

As MD often presents in young patients, some particular assessments may be applied to assess pediatric patients. The Bayley Scales of Infant and Toddler Development-III (Bayley-III) Gross Motor Scale is a functional assessment which can be used to assess muscle strength in subjects with MD ages 2 to <4 years. The minimum score value is 0 and the maximum score value is 72. Higher scores mean a better outcome. The Personal Adjustment and Role Skills Scale, ed. 3 (PARS III) questionnaire is a scale designed to assess behavior and measure psychosocial adjustment of children with chronic physical illnesses. The PARS III is completed by the parent(s)/guardian(s). The minimum score value is 28 and the maximum score value is 112. Higher scores mean better personal adjustment. The Pediatric Outcome Data Collection Instrument (PODCI) measures physical functioning in children. The minimum score value is 0 and the maximum score value is 100. Higher scores mean a better outcome.

In accordance with the treatment methods of the present invention, a therapeutically effective amount/an effective dose of a ROCK inhibitor or a pharmaceutically acceptable salt, solvent or prodrug thereof is administered to a patient one or more times a day. The inhibitor can be administered one time a day, 2 times a day, three times a day, four times a day or more than four times a day.

The following dosing discussion pertains to an adult human subject and dosing in the pediatric population can be reduced using standard conversions based on differences in weight, body volume or surface area. Thus, for example, if a daily dose is 90 mg in a 70 kg adult, this would be 1.29 mg/kg and so a comparable pediatric dose in a 25 kg child would be 32.25 mg. The discussion below pertains to fasudil, but dosing can readily be extrapolated to other ROCK inhibitors described herein based on their relative activity in inhibitor Rho kinases.

The lowest therapeutically effective amount of fasudil, for example, is 90 mg per day, generally administered in 2 to 3 equal portions as an immediate release formulation to obtain the full daily dose. The highest therapeutically effective dose may be determined empirically as the highest dose that remains effective in alleviating one or more MD symptoms but does not induce an unacceptable level or adverse events. Fasudil, for example, generally will not be administered in a daily dose exceeding 240 mg.

Suitable daily doses of fasudil are 90 mg, 100 mg, 110 mg, 1120 mg, 130 mg, 140 mg, 150 mg, 160 mg, 170 mg, 180 mg, 190 mg, 200 mg, 210 mg, 220 mg, 230 mg, or 240 mg. These can be administered in one, two, three, four or more equal doses per day.

As described elsewhere herein the route of administration can be chosen to best suit the requirements. The dosage form can be a dosage form with unmodified release, or it can be a dosage form with modified release. For example, the dosage form can be an immediate-release formulation or an extended release formulation.

One preferred dosing regimen involves the treatment with 60 mg of fasudil hydrochloride hemihydrate three times per day using an oral immediate-release formulation, for a total daily dose of 180 mg. Other daily doses will range from 90 mg to 180 mg per day b.i.d.

A further dosing regimen involves the treatment with 90 mg of fasudil hydrochloride hemihydrate only two times per day using an immediate-release formulation, for a total daily dose of 180 mg.

In other embodiments, fasudil hydrochloride may be administered once a day using an immediate-release formulation at 180 mg or 240 mg. Above 240 mg per day, kidney effects of the drug are generally unacceptable.

Based on ROCK inhibitory activity, one skilled in the art can readily extrapolate the provided dosing ranges for fasudil to other ROCK inhibitors.

Another embodiment involves the treatment with 90 to 240 mg, 120 to 240 mg, 140 to 240 mg, 160 to 240 mg, 180 to 240 mg, 200 to 240 mg, or 220 to 240 mg of fasudil hydrochloride hemihydrate once per day in an extended-release dosage form. Generally, an extended release dosage form will contain from 180 to 240 mg of fasudil hydrochloride hemihydrate. Treatment with an extended-release total daily dose of 180 mg fasudil hydrochloride hemihydrate is preferred.

Methods of administering compositions according to the invention would generally be continued for at least one day, at least 2 days, at least 3 days, at least 4 days, at least 5 days, at least 10 days, at least 15 days, at least 20 days, at least 30 days, at least 40 days, at least 50 days, at least 60 days, at least 70 days, at least 80 days, at least 90 days, at least 4 months, at least 5 months, at least 6 months, at least 7 months, at least 8 months, at least 9 months, at least 10 months, at least 11 months, at least 12 months, at least 18 months, at least 2 years, at least 3 years, or at least 4 years. Some preferred methods treat for up to 30 days or up to 60 days or even up to 90 days or even more. Treatment for more than 60 days is preferred and treatment for at least 6 months is particularly preferred. The precise duration of treatment will depend on the patient's condition and response to treatment. Most preferred methods contemplate that treatment begins after the onset or appearance of symptoms.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

### General Definitions

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Features, integers, characteristics, compounds, chemical moieties or groups described in conjunction with a particular aspect, embodiment or example of the invention are to be understood to be applicable to any other aspect, embodiment or example described herein unless incompatible therewith.

As understood by a person skilled in the medical art, the terms, “treat” and “treatment,” refer to medical management of a disease, disorder, or condition of a subject (i.e., patient) (see, e.g., Stedman's Medical Dictionary). In general, an appropriate dose and treatment regimen provide the inhibitor in an amount sufficient to provide therapeutic and/or prophylactic benefit. Therapeutic benefit for subjects to whom the inhibitor described herein is administered, includes, for example, an improved clinical outcome, wherein the object is to prevent or slow or retard (lessen) an undesired physiological change associated with the disease, or to prevent or slow or retard (lessen) the expansion or severity of such disease. As discussed herein, effectiveness of the inhibitor may include beneficial or desired clinical results that comprise, but are not limited to, abatement, lessening, or alleviation of symptoms that result from or are associated with the disease to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival.

As used herein, the phrase “a disorder associated with muscle fibrosis” refers to but is not limited to any disease or disorder resulting directly or indirectly from and/or completely or partially from muscle fibrosis. Muscle fibrosis can be the origin of the disease or disorder but can also be a symptom appearing after onset of the disease or disorder.

Herein the terms “diseases”, “disorders”, and “conditions” are used interchangeably and refer to a disorder of structure or function in a human or animal, especially one that produces specific symptoms or that affects a specific location and is not simply a direct result of physical injury.

Diseases that can be treated and/or prevented with the inhibitor described herein are described elsewhere herein in detail.

As used herein the term “subject” refers to an individual, e.g., a human, dog, cat, pig, horse, mouse, cow, rat etc having or at risk of having a specified condition, disorder or symptom. The subject may be a patient i.e., a subject in need of treatment in accordance with the invention. The subject may have received treatment for the condition, disorder or symptom. Alternatively, the subject has not been treated prior to treatment in accordance with the present invention. The subject is preferably in need of administration of an inhibitor of the invention.

As used herein, the “administration” or “administering” of a (pharmaceutical) composition described herein to a subject includes any route of introducing or delivering to a subject which allows for the composition to perform its intended function. Administration can be carried out by any suitable route, including orally, intranasally, intraocularly, ophthalmically, parenterally (intravenously, intramuscularly, intraperitoneally, or subcutaneously), or topically. Administration includes self-administration and the administration by another. The composition can be administered as a therapeutically effective amount. As used herein, the phrase “therapeutically effective amount” means a dose or plasma concentration in a subject that provides the specific pharmacological effect for which the described compositions are administered, e.g., to treat a disease of interest in a target subject. The therapeutically effective amount may vary based on the route of administration and dosage form, the age and weight of the subject, and/or the disease or condition being treated.

A composition may be a pharmaceutical composition or formulation that comprises the inhibitor and a pharmaceutically acceptable excipient, adjuvant, diluent and/or carrier. Pharmaceutical compositions or formulations may routinely contain pharmaceutically acceptable concentrations of salt, buffering agents, preservatives, compatible carriers, supplementary immune potentiating agents such as adjuvants and cytokines and optionally other therapeutic agents or compounds.

As used herein, “pharmaceutically acceptable” refers to a material that is not biologically or otherwise undesirable, i.e., the material may be administered to an individual along with the selected inhibitor without causing any undesirable biological effects or interacting in a deleterious manner with any of the other components of the pharmaceutical composition in which it is contained.

Excipients are natural or synthetic substances formulated alongside an active ingredient (e.g. a neurotoxin as provided herein), included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, glycerol, ethanol, and the like.

Adjuvants are pharmacological and/or immunological agents that modify the effect of other agents in a formulation. Pharmaceutically acceptable adjuvants are well known in the art and include cell-penetrating peptides. A suitable adjuvant is therefore easily identifiable by one of ordinary skill in the art.

Diluents are diluting agents. Pharmaceutically acceptable diluents are well known in the art and include water or saline. A suitable diluent is therefore easily identifiable by one of ordinary skill in the art.

Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art and include serum albumin. A suitable carrier is therefore easily identifiable by one of ordinary skill in the art.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. It is to be understood that this invention is not limited to the particular methodology, protocols, and reagents described, as these may vary, depending upon the context they are used by those of skill in the art.

The term “alkyl” as used herein, by itself or in conjunction with another term or terms, refers to a branched or unbranched saturated hydrocarbon chain. The alkyl group is described using a prefix designating the minimum and maximum number of carbon atoms in the moiety, e.g. “C_{a-b}”. For example, C_{a-b}alkyl indicates an alkyl moiety having the integer “a” to the integer “b” number of carbon atoms, inclusive. Unless specified otherwise, alkyl groups are unsubstituted. Representative examples include, but are not limited to, methyl, ethyl, n-propyl, i-propyl, n-butyl, i-butyl, s-butyl, t-butyl, n-pentyl and n-hexyl.

The term “haloalkyl” as used herein refers to an alkyl group in which one or more hydrogen atoms are replaced by halogen atoms. The alkyl group in “haloalkyl” is a saturated alkyl group. The alkyl group is described using a prefix designating the minimum and maximum number of carbon atoms in the alkyl moiety. Representative examples of “haloalkyl” include, but are not limited to, —CF₃, —CHF₂, —CH₂F, —CH₂Cl, —CH₂Br, —CF₂CF₃, —CHFCF₃, —CH₂CF₃, —CF₂CH₃, —CHFCH₃, —CF₂CF₂CF₃, —CF₂CH₂CH₃, CHFCH₂CH₃ and —CHFCH₂CF₃. Haloalkyl groups are unsubstituted.

The term “heterocycloalkyl” as used herein, by itself or in conjunction with another term or terms, refers to a monocyclic, non-aromatic ring systems, which contains at least one nitrogen atom (as shown in formula (1)) and carbon atom(s). In addition to the nitrogen atom, the heterocycloalkyl group may include one or more additional heteroatoms, such as nitrogen, oxygen, sulfur or phosphorus, preferably nitrogen, oxygen or sulfur, particularly nitrogen or oxygen. Heterocycloalkyl groups may be fully saturated or contain unsaturated portions. In some instances, a heterocycloalkyl group may contain a single nitrogen heteroatom (e.g. as shown in formula (1)) or at least two or heteroatoms, which may be the same or different. Heterocycloalkyl groups can be substituted or unsubstituted. In some instances, a heterocycloalkyl group may contain 5 ring atoms, 6 ring atoms, or 7 ring atoms.

The term “hydroxyalkyl” as used herein refers to an alkyl group in which one or more hydrogen atoms are replaced by hydroxy groups. The alkyl group in “hydroxyalkyl” is a saturated alkyl group. The alkyl group is described using a prefix designating the minimum and maximum number of carbon atoms in the alkyl moiety. Representative examples of “hydroxyalkyl” include, but are not limited to, —CH₂OH, —CH₂CH₂OH, —CH₂CH(OH)CH₃, —CH₂CH₂CH₃, OH. Hydroxyalkyl groups are unsubstituted. In general, it is preferred that the hydroxyalkyl group has a single hydroxy group.

The term “pharmaceutically acceptable salt” as used herein refers to a salt of compound, particularly a compound of formula (1), that is generally chemically and/or physically compatible with the other ingredients comprising a formulation, and/or are generally physiologically compatible with the recipient thereof. A suitable pharmaceutically acceptable salt of a compound of formula (1) is, for example, an acid addition salt of the compound, such as an acid addition salt of an inorganic or organic acid. Examples of such acids include hydrochloric, hydrobromic, sulfuric, phosphoric, trifluoroacetic, formic, citric or maleic acid. In some instances, the compound of formula (1) may be acidic and can form a salt with a counter cation. The salt may an alkali metal salt (e.g. sodium or potassium salt), an alkaline earth metal salt (e.g. a calcium or magnesium salt), an ammonium salt or a salt with an organic base which affords a physiologically acceptable cation (e.g. a salt with methylamine, dimethylamine, trimethylamine, piperidine, morpholine or tris (2 hydroxyethyl) amine). In general, a pharmaceutically acceptable salt of a compound of formula (1) is an acid addition salt.

Representative salts include, but are not limited to, acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate and the like. Other examples of representative salts include alkali or alkaline earth metal cations such as sodium, lithium, potassium, calcium, magnesium, and the like, as well as non-toxic ammonium, quaternary ammonium and amine cations including, but not limited to, ammonium, tetramethylammonium, tetraethylammonium, lysine, arginine, benzathine, choline, tromethamine, diolamine, glycine, meglumine, olamine and the like.

The compounds of formula (1) may exist in solvated (e.g. hydrated form) or unsolvated forms. It is to be understood that the invention encompasses all such solvated forms that possess RhoA/ROCK pathway inhibitory activity.

The compounds of formula (1) may be administered in the form of a pro drug which is broken down in the human or animal body to release the compound. A pro-drug may be used to alter the physical properties and/or the pharmacokinetic properties of the compound. A pro-drug can be formed when the compound of formula (1) contains a suitable group or substituent to which a property-modifying group can be attached. Examples of pro-drugs include in vivo cleavable ester derivatives that may be formed at a carboxy group or a hydroxy group in a compound of the formula (1), in-vivo cleavable amide derivatives that may be formed at a carboxy group or an amino group in a compound of the formula (1) or an N-oxide formed at an amine moiety in a compound of the formula (1).

The reference to a pro-drug of the compound of formula (1) may be an ester, an amide or an N-oxide thereof.

Aspects of the invention are demonstrated by the following non-limiting examples.

### Examples

The invention will now be further described with reference to the following Examples and accompanying figures.

### Results

### Pdgf-AA Induces Activation of Rho-a Pathway in Human Muscle FAPs

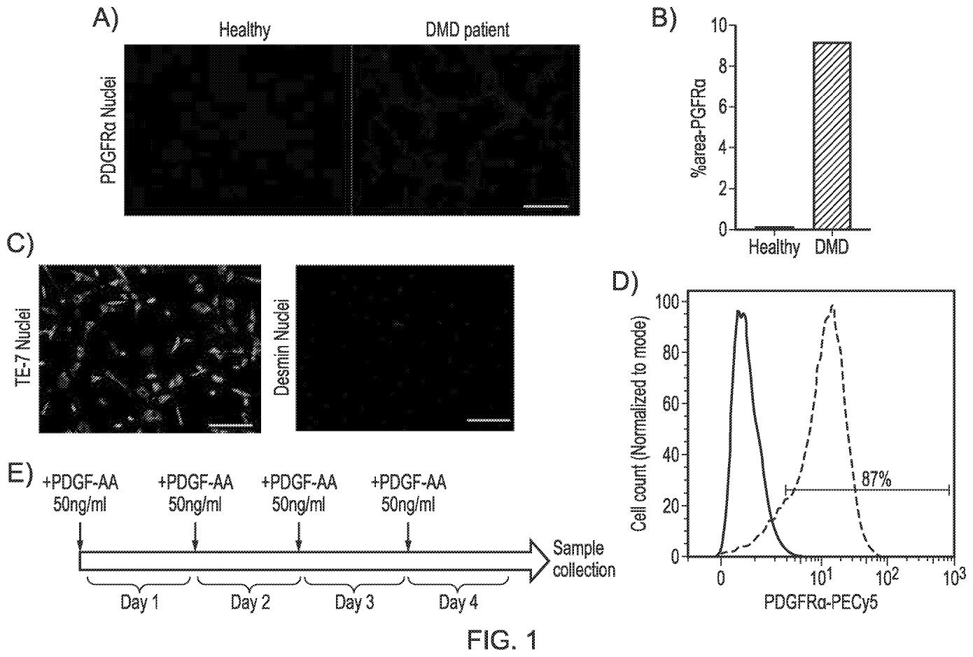

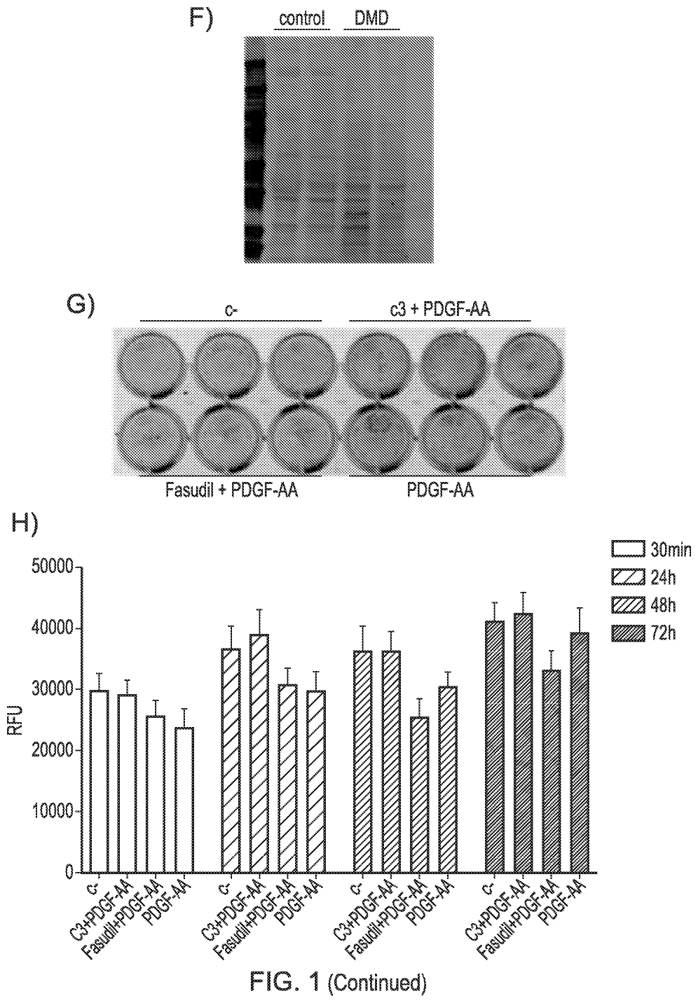

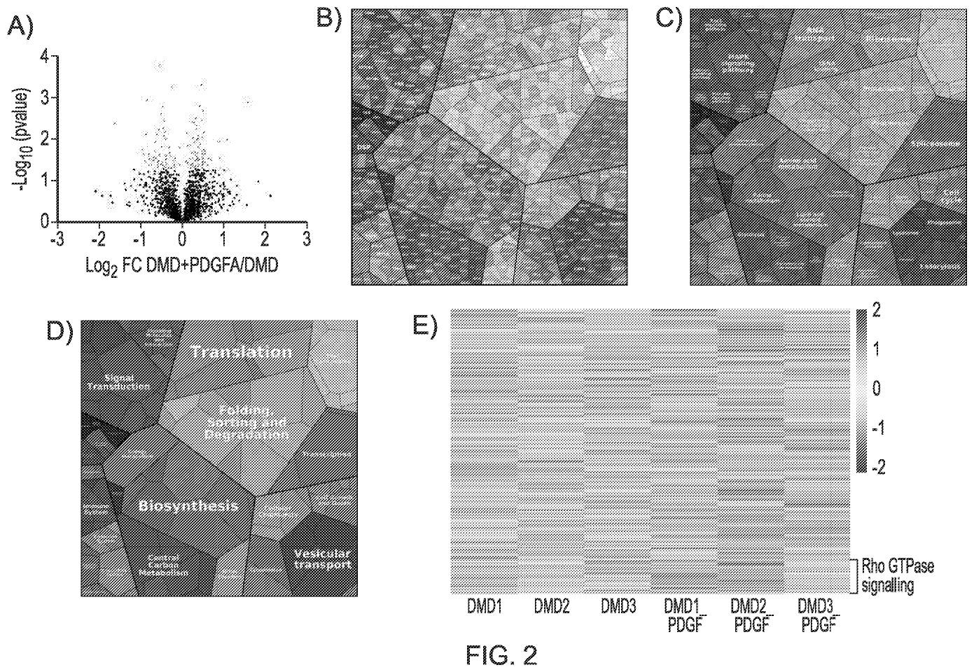

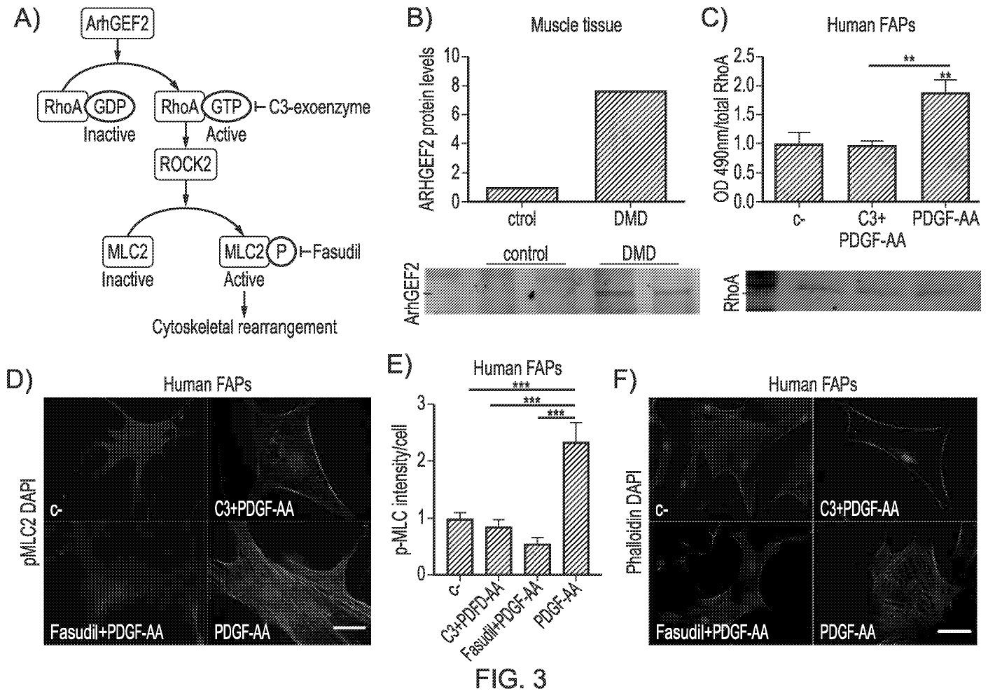

First it was confirmed that the number of FAPs was increased in DMD muscle biopsies (n=2) compared to healthy controls (n=2) (FIGS. 1A-B). Then, FAPs were isolated from muscle biopsies of 3 DMD patients and treated them with 50 ng/ml of PDGF-AA for 4 days. To analyse changes in the protein profile, the inventors used quantitative proteomic analysis with mass spectrometry comparing PDGF-AA treated FAPs and non-treated FAPs. It was observed that 1890 proteins were differentially expressed (Volcano plot in FIG. 2A). The inventors built a proteomap of the upregulated proteins according to the functional gene classification (Kyoto Encyclopedia Genes and Genomes (KEGG)) (FIG. 2B). An increase in the expression of proteins involved in various signalling pathways (MAPK, PI3K-Akt, FoxO or Hedgehog), cell metabolism and genetic information processing (FIG. 2C-D) was observed. Among the different protein groups that were upregulated, the inventors were interested in those involved in cell functions related to fibrosis such as cytoskeletal rearrangement, cell adhesion, and rearrangement of actin filaments. Proteins related to these cellular functions, such as proteins of the Ras homolog gene pathway (Rho), were increased in FAPs treated with PDGF-AA as displayed in a heatmap showing the differences between DMD-FAPs untreated and treated with PDGF-AA (FIG. 2E). Among the different proteins of this family, it was focused on the Ras homolog gene family member A (RhoA) pathway since it has been involved in fibroblast activation and fibrosis in several other pathologies.

### C3 Exoenzyme and Fasudil Treatment Block Rho-Kinase Pathway Activation Mediated by PDGF-AA.

Mass spectrometry results were validated in two different experiments. First, the inventors studied the expression of guanine nucleotide exchange factor Rho 2 (ArhGEF2), which is the effector protein in the Rho-kinase pathway (FIG. 3A), in DMD muscle samples and aged and sex matched controls (loading control in FIG. 1E). A 7.7-fold increase in the expression of ArhGEF2 in DMD muscles compared to control muscles (n=2) (FIG. 3B) was observed. In a second step it was analysed if the addition of PDGF-AA to DMD-FAPs in culture induced a significant increase in RhoA bound to guanosine triphosphate (GTP) (RhoA-GTP) compared to untreated FAPs (c-). To further confirm whether the increase in RhoA-GTP was mediated via activation of RhoA pathway, the effect of pre-treatment with the exoenzyme C3 (C3) was also analysed. Exoenzyme C3 is an ADP ribosyl transferase that selectively ribosylates RhoA proteins at asparagine residue 41, rendering it inactive. As expected, PDGF-AA treatment induced a significant 1.88-fold increase in the RhoA-GTP levels (n=3, SEM=0.206, p<0.01) while pre-treatment with C3 blocked the effect induced by PDGF-AA (n=3, SEM=0.07, p<0.05) (FIG. 3C). Treatment with PDGF-AA, C3 or fasudil did not affect cell viability at any time tested (FIG. 1H).

Fasudil is a commercially available vasodilator used to treat cerebral vasospasm but also useful for pulmonar hypertension. Fasudil inhibits RhoA pathway by blocking the Rho-associated protein kinase (ROCK) which phosphorylates myosin light chain 2 (p-MLC2). Treatment of DMD-FAPs with PDGF-AA induced a statistically significant 2.3-fold increase in MLC2 phosphorylation (n=3, SEM=0.33, p<0.001), which was reversed by pre-treatment with C3-exoenzyme (n=3, SEM=0.11, p<0.001) and fasudil (n=3, SEM=0.09, p<0.001), as shown in FIGS. 3D and 3E. The last step on the Rho-kinase pathway is the polymerization of F-actin filament mediated by MLC2 phosphorylation. Treatment of FAPs with PDGF-AA induced polymerization on intracellular actin filaments, which was impinged by pre-treatment with C3 or fasudil (FIG. 3F).

### Fasudil Blocks PDGF-AA Mediated Increase in Proliferation, Migration and Expression of Collagen-I by Human FAPs In Vitro.

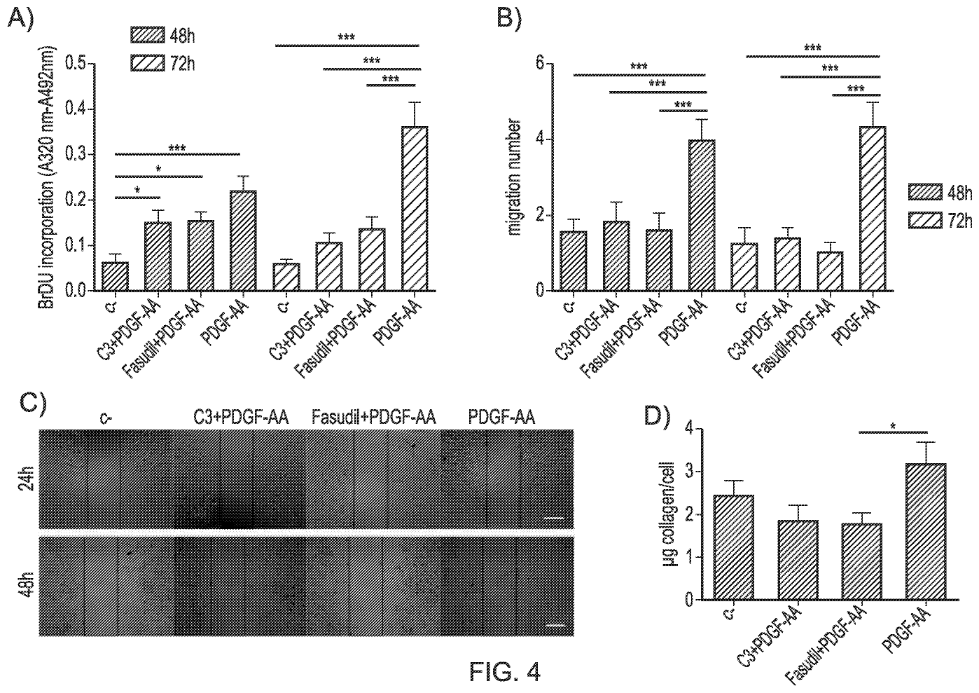

The effect of PDGF-AA, C3-exoenzyme and fasudil in proliferation, migration, and collagen-l production was studied in vitro. Addition of PDGF-AA to the culture medium significantly increased FAPs proliferation at both 48 (n=3, SEM=0.02, p<0.001) and 72 hours (n=3, SEM=0.05, p<0.001) (3.6-fold and 6-fold increase, respectively) compared with untreated FAPs. In contrast, treatment of FAPs with C3-exoenzyme (n=3, SEM=0.01, p<0.001) and fasudil (n=3, SEM=0.02, p<0.001) prior to stimulation with PDGF-AA lead to a significant decrease in proliferation rate (3.27-fold and 2.6-fold decrease, respectively) compared to FAPs treated with PDGF-AA at 72 hours (FIG. 4A). All treatment doses and the different times of cell exposure were tested for cell viability. PrestoBlue data showed that neither C3-exoenzyme nor fasudil had any effect on cell viability (FIG. 1H).

Treatment with PDGF-AA significantly enhanced FAPs migration in vitro compared to untreated FAPs at both 48 (n=3, SEM=0.51, p<0.001) and 72 hours (n=3, SEM=0.66, p<0.001) (2.48-fold and a 3.37-fold increase respectively). This effect was significantly blocked when C3-exoenzyme and fasudil were added to the medium (FIG. 4B-C) either at 48 or 72 hours. C3-exoenzyme reduced migration rate 2.1-fold at 48 hours (n=3, SEM=0.47, p<0.001) and 3-fold at 72 hours (n=3, SEM=0.25, p<0.001). Fasudil reduced migration rate 2.43-fold at 48 hours (n=3, SEM=0.41, p<0.001) and 4-fold at 72 hours (n=3, SEM=0.22, p<0.001).

PDGF-AA treatment also increased non-significantly the release of collagen-I to the culture medium an effect that was reversed by the addition of C3-exoenzyme and fasudil (FIG. 4D). C3-exoenzyme significantly reduced the release of collagen-I by 1.7 fold (n=3, SEM=0.33, p<0.05).

Fasudil Treatment Improves Muscle Function and Reduces Muscle Fibrosis in the dba/2J-mdx Murine Model of DMD.

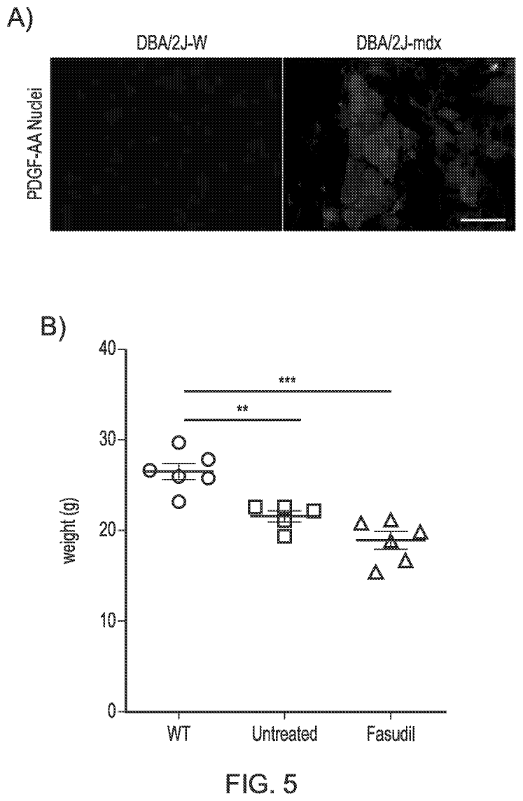

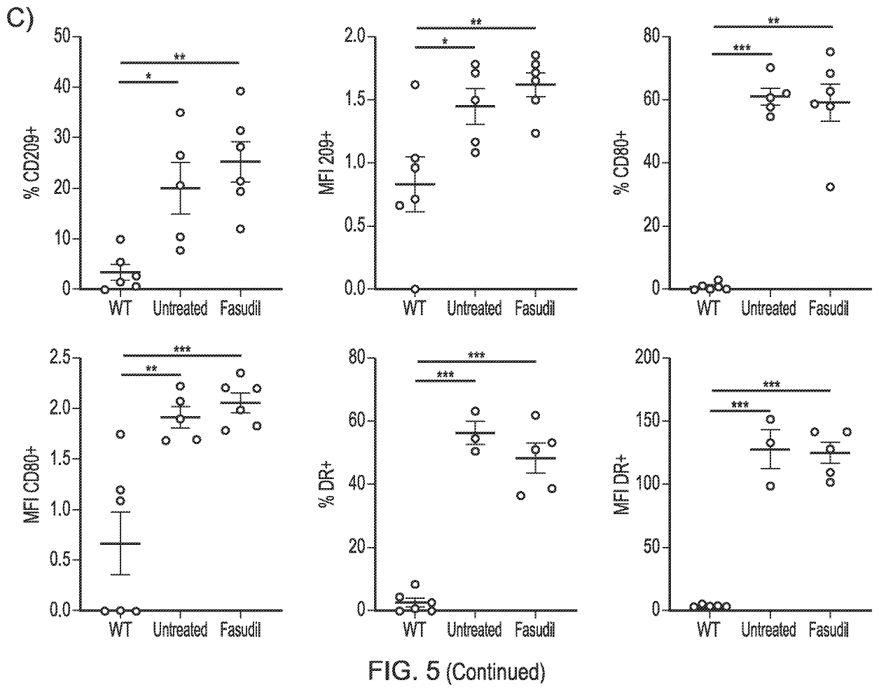

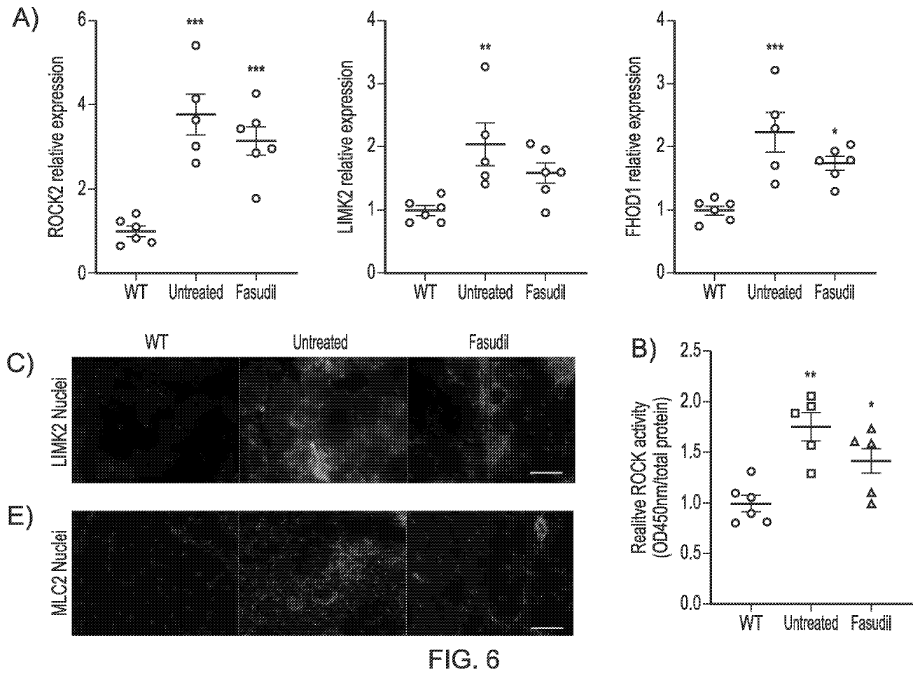

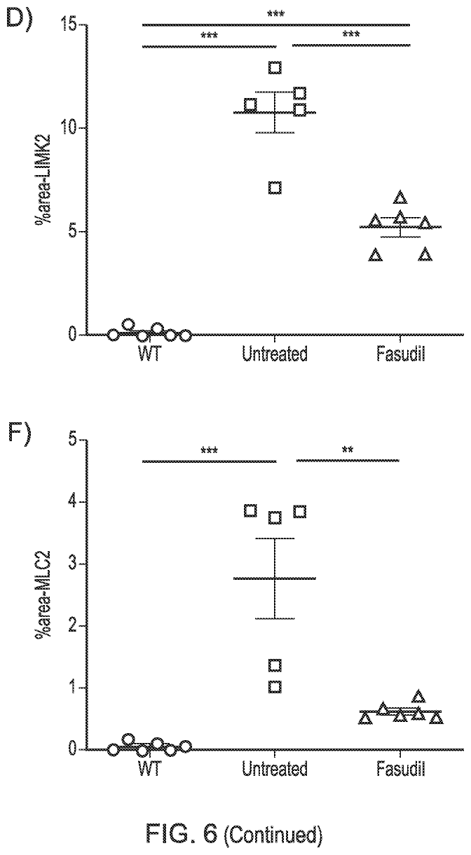

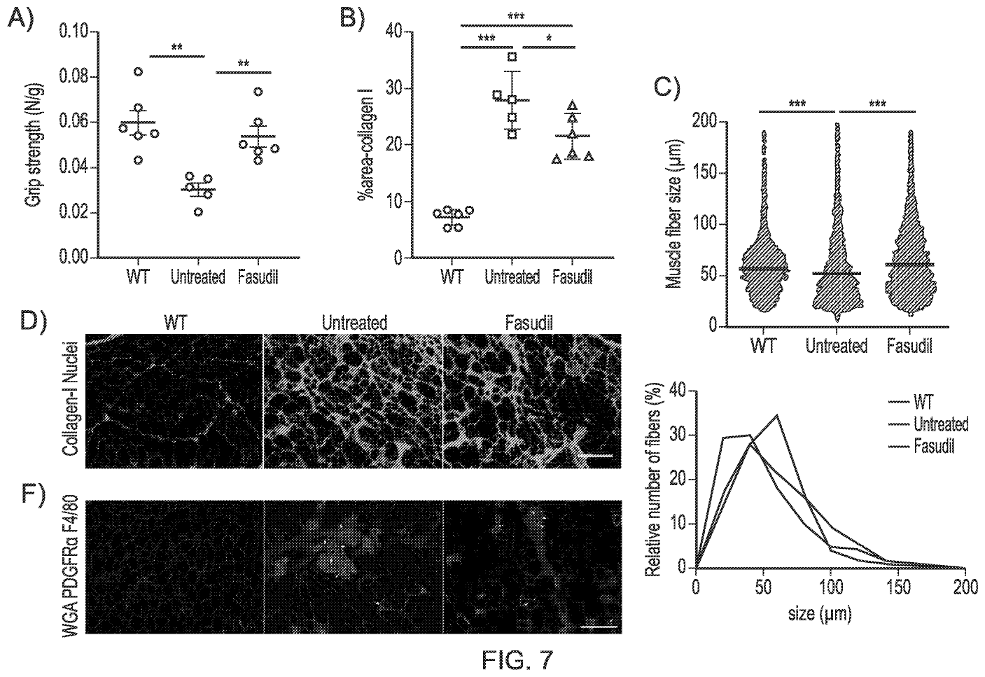

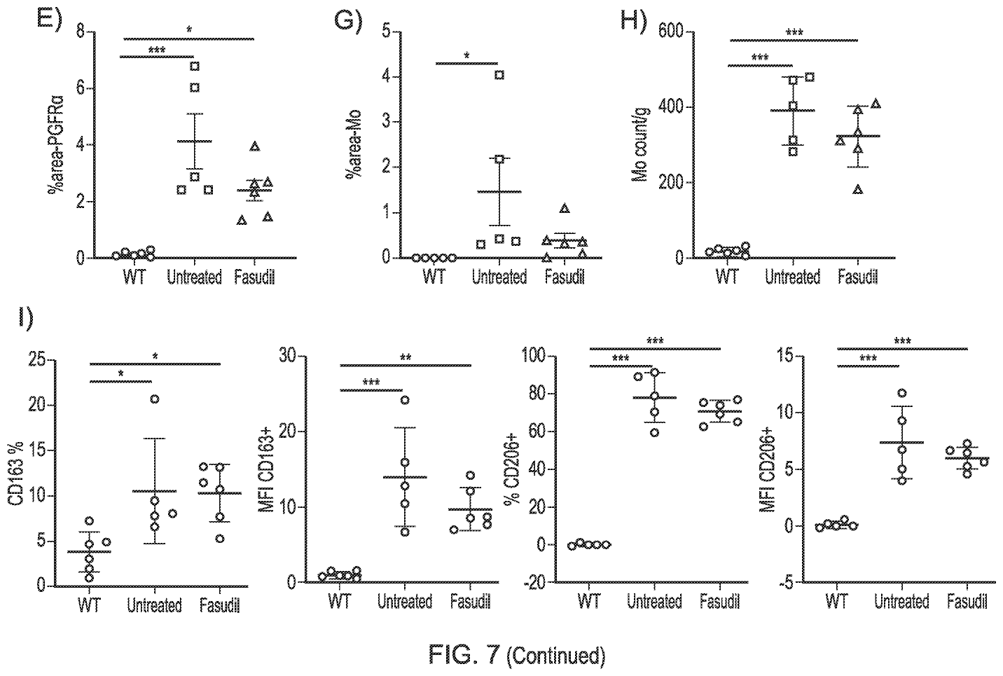

Based on the in vitro results it was decided to test if fasudil had an antifibrotic effect in vivo in the dba/2J-mdx murine model of DMD. First it was confirmed that PDGF-AA levels were increased in muscle samples of dba/2J-mdx mice compared to healthy WT controls (FIG. 5A) as happens in DMD patients. Based on these results 6 dba/2J-mdx mice of 7 weeks old were treated with fasudil at a dose of 100 mg/kg/day orally for 6 weeks. Mice did not experience any change in their behaviour during the treatment period suggesting that there were not significant adverse effects. However, a decrease in weight was observed when compared treated (n=6, SEM=0.94) and untreated mice (n=5, n=0.60) (FIG. 5B) although these differences did not reach statistical significance. It was confirmed that fasudil inhibited RhoA pathway by analysing different downstream effector targets of RhoA, such as ROCK2, LIM domain kinase 2 (LIMK2) and Formin Homology 2 Domain Containing 1 (FHOD1), being the two later proteins involved in the regulation of actin filament dynamics (FIG. 6A). The inventors also quantified the enzymatic activity of ROCK, which was decreased in quadriceps in treated mice (n=6, SEM=0.12) compared to non-treated mice (n=5, SEM=0.14), although these changes were non-significant (FIG. 6B). However, the protein expression of LIMK2 and MLC2 was significantly reduced in treated (n=6, SEM=0.45 and n=6, SEM=0.05, respectively) compared to untreated mice (n=5, SEM=0.97 and n=5, SEM=0.64, respectively) (FIG. 6C-F). Muscle strength was analysed using forelimb grip-strength after six weeks of treatment and compared the results obtained in non-treated dba/2J-mdx and healthy control mice. A significant 1.76-fold increase was observed in forelimb strength in treated mice (n=6, SEM=1.63) compared with non-treated mice (n=5, SEM=2.29, p<0.01) (FIG. 7A). Interestingly, differences between treated mice and WT mice did not reach significance. The inventors also analysed the effect of fasudil on quadriceps histology. Fasudil induced a significant 23% reduction in collagen-I area in the quadriceps of treated mice (n=6, SEM=1.63, p<0.05) (FIGS. 7B and D). A significant increase in the myofiber size of treated mice (n=6, SEM=0.60) compared to non-treated (n=5, SEM=0.64, p<0.001) was detected (FIG. 7C). Additionally, a 42% decrease was observed in the PDGFRα area as a measure of the number of FAPs present in the quadriceps of treated mice (n=6, SEM=0.38) compared to non-treated mice (n=5, SEM=0.95), although these differences did not reach significance (FIGS. 7E and F).

### Effect of Fasudil Treatment in Inflammation

Quantification of macrophages was performed by analysing the area of the quadriceps stained with F4/80 maker. Fasudil induced a 74% reduction in treated mice (n=6, SEM=0.15) when compared to untreated mice (n=5, SEM=0.73) (FIGS. 7F and G). Those results were in accordance with flow cytometry analysis which showed a 18.3% decrease in the number of F4/80 positive cells in treated mice (n=6, SEM=33.49) compared to non-treated mice (n=5, SEM=39.82) (FIG. 7H). Indeed, a trend towards a reduction in the expression of profibrotic M2 markers, such as CD163 and CD206, was observed in macrophages obtained from fasudil treated mice. In detail, a 31% reduction was observed in the expression levels of CD163 marker in macrophages obtained from skeletal muscles of treated mice (n=6, SEM=1.10) although there was only a 3% lower number of CD163+ cells compared to non-treated mice (n=5, SEM=2.58). Similarly, a 18% reduction was observed in the expression levels of CD206 marker in macrophages obtained from treated mice (n=6, SEM=1.43) although there was only a 10% lower number of CD206+ cells compared to non-treated mice (n=5, SEM=5.91) (FIG. 7I). The inventors did not observe any difference either in the expression levels or in the number of CD209 cells which also identify M2 macrophages. The inventors did not observe changes in the percentage of positive cells or in the expression of CD80 and DR, two well-known M1 markers, in the macrophages obtained from skeletal muscles of treated mice compared to non-treated mice (FIG. 5C).

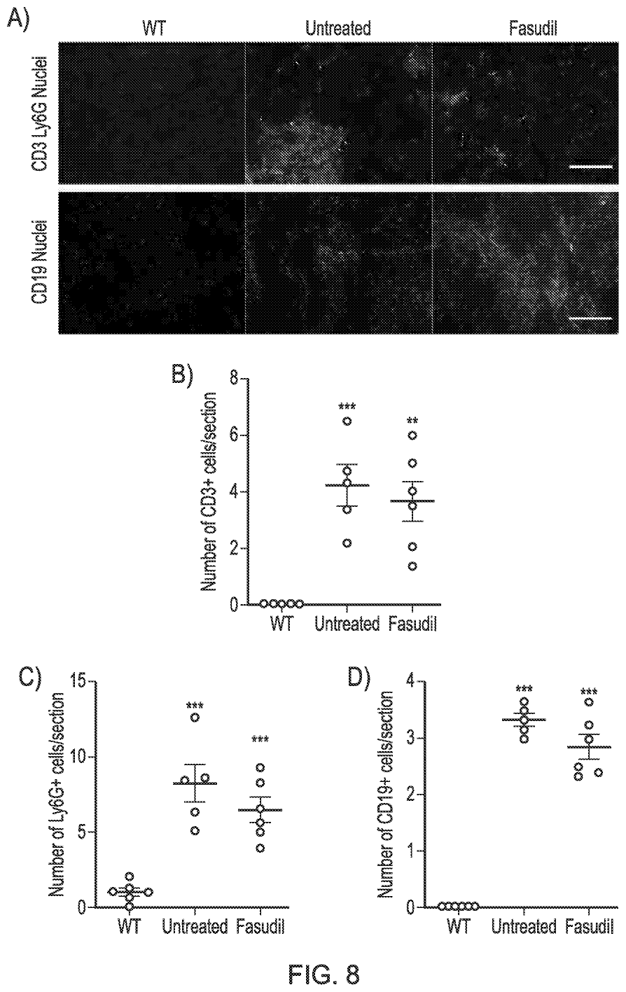

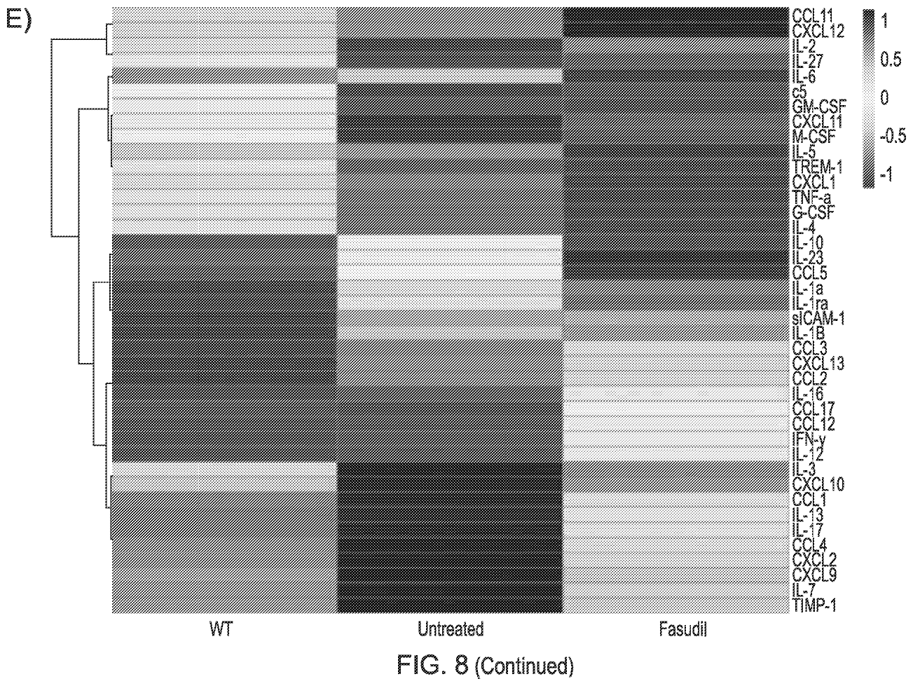

Also the population of T cells, B cells and neutrophils was studied and a trend towards a reduction in the neutrophil population in treated mice (n=6, SEM=0.72) compared to non-treated (n=5, SEM=0.74) was observed, while there were no changes in the number of T and B cells (FIG. 8A-D). Finally, it was studied if fasudil treatment modified the expression of several cytokines involved in the inflammatory process in muscular dystrophies. As shown in a heatmap in FIG. 8E, a reduction in TNFα, IL-4, IL-17, CXCL1, CXCL2, CXCL10, CCL2, CCL3 or CCL17 among others in fasudil treated mice when compared to non-treated mice was observed.

### DISCUSSION

In this study the inventors have investigated the molecular pathways activated by PDGF-AA in skeletal muscles of patients with DMD driving to muscle fibrosis. The inventors have observed that PDGF-AA activates RhoA/ROCK2 pathway which can be effectively blocked by fasudil, a well-known Rho-kinase inhibitor that reduces FAPs activation in vitro. A proof-of-concept preclinical study was performed to validate these findings observing a reduction in muscle fibrosis in the murine model of DMD. The results expand the molecular pathways involved in the process of muscle fibrosis in DMD and support the efficacy of Rho-kinase inhibitors as a treatment of DMD in particular and muscular dystrophies in general.

Absence of dystrophin in DMD produces sarcolemma instability leading to expansion of fibrotic and fat tissue. Several growth factors have been related to the fibrotic expansion; however the role of PDGF-AA has not been fully studied. Although previous studies found that the signalling through PDGFRα is persistent in DMD leading to fibrosis and hindering repair, the molecular consequences of persistent exposure of FAPs to PDGF-AA have not been addressed. To study the molecular changes that occurs in DMD, FAPs from DMD patients were treated with PDGF-AA and observed an upregulation of several proteins belonging to the RhoA/ROCK2 pathway. The RhoA/ROCK2 signalling pathway is well known for regulating actin cytoskeleton organization and cellular dynamics in several cell types. RhoA acts through two molecular conformations: it is inactive when bound to a guanosine diphosphate (GDP), and active when bound to GTP. ROCK2, the Rho downstream effector molecule, is a serine/threonine kinase protein that regulates actin filament remodelling by phosphorylating numerous downstream target proteins, including the myosin binding subunit of myosin light chain 2 (MLC2) phosphatase. Actin reorganization triggered by RhoA/ROCK2 signalling pathway drive fibroblasts activation in different pathologic conditions such as cardiac fibrogenesis, tumor-cell growth or pulmonary artery hypertension. Actin polymerization regulates cell polarization, organization of adhesion structures and the generation of the force essential for cell migration. These events allow cells to migrate into the injured site where they proliferate and release components of the ECM to regenerate the damaged tissue. However, an excessive activation of fibroblasts increases the ECM deposition leading to accumulated fibrogenesis. Although the rearrangement of actin produced in fibroblast activation has not been characterized in FAPs, previous studies suggest that fibroblasts and FAPs are phenotypically and biochemically equivalent supporting that Rho-kinase activation could have a similar effect in both cells. The inventors demonstrated that RhoA/ROCK2 pathway is increased in muscles of DMD patients when compared to age and sex matched healthy controls subjects. Therefore, it was decided to analyse the effect of PDGF-AA on RhoA/ROCK2 signalling pathway in FAPs isolated from DMD patients. First, it was confirmed that RhoA/ROCK2 pathway can be modulated by PDGF-AA since C3-exoenzyme and fasudil significantly reduced the signalling pathway activation. The results show that PDGF-AA activated RhoA/ROCK2 pathway resulting in an increase of actin filament polymerization, proliferation and migration and that both C3 and fasudil attenuated these effects. Although PDGF-AA induced a higher proliferation rate at 48 and 72 hours, the blocking effect of RhoA/ROCK2 inhibitors was only observed at 72 hours. The lack of proliferation inhibition at 48 hours after C3-exoenzyme or fasudil treatment could be due to other signalling pathways activated after PDGF-AA treatment. PDGF-AA is a mitogen growth factor that not only activates RhoA pathway but also triggers other signalling pathways, such as MAPK, PLC or PI3K. While these molecular pathways are mainly involved in cell proliferation, RhoA/ROCK2 is involved in actin remodelling.

Since the Rho/ROCK-mediated pathway interacts with other signaling pathways known to contribute to fibrosis, it was tested whether fasudil reduces fibrosis in the dba/2J-mdx mice. Fasudil is a selective inhibitor of ROCK1 and ROCK2 isoforms that has been used in clinic as a first-generation selective Rho/ROCK inhibitor. Fasudil has been tested in different diseases such as pulmonary hypertension, amyotrophic lateral sclerosis or cardiovascular disease. The inventors observed that dba/2J-mdx mice treated with fasudil had better performance on grip strength test than non-treated mice. These results were associated to a decrease in the amount of collagen-I and to a tendency towards a decreased number of FAP cells in the skeletal muscles of the treated animals compared to the non-treated ones.

Since the inflammatory response is another characteristic feature of DMD muscles, the inventors also analysed inflammatory cells profile after treatment. Fasudil induced a trend towards a reduction in the number of neutrophils and macrophages, particularly in the number of CD163 and CD206 positive macrophages without modifying M1 population. Additionally, fasudil reduced the expression of several inflammatory cytokines, some of them directly involved in the fibrotic process such as IL4, IL17 or CCL17. Although previous studies have shown that RhoA can be expressed in macrophages, the inventors just observed a non-significant reduction in their number when blocking this pathway with fasudil. The changes that have been observed in the number of infiltrating inflammatory cells and in the reduction of the cytokine levels could be explained either by a direct effect of fasudil on inflammatory cells or by an indirect effect of the reduced fibrosis content of the muscle. It is well known that continuous muscle damage perpetuates the inflammatory response in muscular dystrophies and therefore, treatments improving the general architecture of the muscles reducing muscle degeneration can have an indirect effect reducing inflammation. For example, the attenuation of the fibrotic process in the muscle may improve muscle regeneration in treated animals reducing the number of degenerating fibers and preventing macrophages and neutrophils to infiltrate the injured muscle. Indeed, the increased fiber size observed in treated mice could be due to a better regeneration of the muscle together with a less affected muscle architecture. In summary, treatment with fasudil on dba-2J mdx mice improved grip-strength test, reduced expression of collagen-I and resulted in a trend towards a decreased number of PDGFRα+ cells on skeletal muscles, confirming the results in vitro. The results suggest that these effects are mainly explained by the effect that RhoA/ROCK2 inhibition has on fibrosis deposition.

Although ROCK is expressed in different cell types, side effects produced by fasudil does not include major safety concerns. Fasudil targets the ATP-dependent kinase domain of either ROCK1 and ROCK2 with equal potency and without selective effects. It is worth noting that ROCK2 is the isoform detected in the inventors' proteomic analysis and previous studies have shown that ROCK2 is the predominant isoform in skeletal muscle.

Muscle fibrosis is an irreversible process that takes place in muscular dystrophies and should be prevented before the accumulation of ECM disturbs muscle architecture and function. Since FAP activation and ECM remodelling start early during skeletal muscle degeneration, inhibiting the initial cellular changes that occur on FAPs could prevent the increase of ECM deposition. Therapies focused on targeting actin rearrangement to prevent FAP migration into the injury site may help to slow down the progression of fibrosis. Coupling anti-fibrotic therapies with cell therapy or gene therapy could also lead to a better outcome of these experimental therapies.

In summary, this study demonstrates that PDGF-AA induces RhoA/ROCK2 pathway signalling in DMD-FAPs leading to their proliferation, migration and actin reorganization. Treatment with fasudil, a well-known ROCK inhibitor, blocks the effect of PDGF-AA on cells in vitro and reduces muscle fibrosis increasing muscle strength in a DMD murine model.

### Material & Methods

### Cell Culture

Muscle biopsies from 3 DMD patients seen at Hospital de la Santa Creu i Sant Pau (HSCSP) in Barcelona (Table 1) were used. The Ethics Committee of HSCSP approved the study, and all participants signed an informed consent form. All study procedures were performed in accordance with Spanish regulations.

Muscle explants were cultured and FAPs were isolated as described herein. FAPs were treated with PDGF-AA at 50 ng/ml each day for 4 days (FIG. 1E). To test the activation of the RhoA pathway with PDGF-AA, the pathway was first inhibited with fasudil 50 μM (Bio-Techne, Minnesota, USA) or C3-exoenzyme 2 μg/ml (Cytoskeleton, Denver, USA) for 15 hours and then activated with PDGF-AA at 50 ng/ml for 20 minutes and proceed to the G-LISA or IF assay of myosin light chain phosphorylation (p-MLC). To test the functional effect of the PDGF-AA pathway and its inhibition, C3-exoenzyme at 2 μg/ml or fasudil at 50 μM was first added to the culture and then PDGF-AA was added 4 hours later at 50 ng/ml. This inhibition/induction cycle was repeated for as many days as the test lasted.

### Quantitative Proteomics

The protein samples were extracted and analysed using a Lumos Orbitrap mass spectrometer (Thermo Fisher Scientific, San Jose, CA, USA) coupled to an EASY-nLC 1000 (Thermo Fisher Scientific (Proxeon), Odense, Denmark). The samples were analysed with the MaxQuant software (version 1.6.1.0) through the human Swissprot database.

### Cell Viability

The effect of each treatment on FAP viability was measured with the PrestoBlue reagent (Invitrogen) following manufacturer's instructions. Fluorescence was measured at wavelengths 570 nm excitation and 600 nm emission using a microplate reader (INFINITE M1000 PRO, Tecan Trading AG, Switzerland).

### G-LISA, ROCK Activity, Sircol Assay and Cytokine Array

The RhoA-GTP protein was analysed by a colorimetric assay using the G-LISA assay (Cytoskeleton). The enzymatic activity of ROCK was assessed by a colorimetric method by using a ROCK activity assay (Abcam) in protein extract from quadriceps. Total soluble collagen released by FAPs was measured using Sircol kit (Biocolor, UK). Mouse cytokines were determined using the Proteome Profiler Cytokine Array Panel A (R&D Systems, USA) (see supporting information).

### Proliferation and Migration Assays

The proliferation assay was carried out following the manufacturer's instructions (Roche, Indianapolis, IN) at 24 and 72 hours. The migration assay was performed using 24-well plates with inserts (Ibdi, Munich, Germany) at 48 and 72 hours.

### Western-Blot

Cells corresponding to each condition or muscle tissue were lysed in RIPA buffer (Sigma-Aldrich) with protease and phosphatase inhibitors (Roche, Basel, Switzerland) and the specific bands corresponding to the proteins of interest were visualized with the Odyssey infrared detection system (Li-Cor) together with the Image Studio software (Li-Cor). Total protein bands were measured with the Revert 700 kit (Li-Cor) (FIG. 1F). The primary and secondary antibodies used in the study are listed in table 1.

### Histology and Immunofluorescence

Cultured FAPs were fixed with ethanol and incubated with blocking solution (Santa Cruz Biotech, Dallas, TX, USA)). Frozen muscle sections were obtained with a cryostat (Leica Microsystems, Wetzlar, Germany), fixed with acetone and incubated with blocking solution (Santa Cruz Biotech). The primary and secondary antibodies used in the study are listed in table 2.

Images were obtained with an Olympus BX51 microscope coupled to an Olympus DP72 camera. Image J software was used to quantify the intensity of individual cells and the area of positive staining in muscle tissue. A minimum of five independent fields per staining were quantified.

### Mouse Model

Animal procedures were performed according to the National Institutes of Health Guidelines for the Care and Use of Laboratory Animals and were approved by the Ethical Committee of the Universitat Autonoma de Barcelona. The animals used in this study were all 7-week old males. Six dba/2J-mdx males were treated with fasudil (LC Laboratories) that was diluted in water and administered at 100 mg/kg/day orally for 6 weeks by gavage, according to previous studies. Five untreated dba/2J-mdx males were used as controls and six dba/2J males were used as healthy controls.

### Grip Strength Test

Force was measured using a Grip Strength Meter. Mice were initially held by the tail above the grid. Once mice grasped the grid, they were pulled out until release. The procedure was repeated 5 times and the mean of the 3 highest values was used. Mean result was normalized to the body weight of the animal and is presented as grip force/weight (N/g).

### Flow Cytometry

Single cell suspension from cell culture, quadriceps and tibial anterior were obtained for flow cytometry. Cultured human muscle derived cells were labelled using anti-PDGFRα-followed by streptavidine-PECy5 and anti-CD56. Cells obtained from mice were labelled using anti-sca1, anti-CD45, anti-F4/80, anti-CD163 and a viability marker (table 1). Samples were acquired with the MACSQuant Analyzer 10 flow cytometer (MiltenyiBiotec). Doublet cells were excluded using Forward scatter area and height. Compensations were adjusted according to the single stained controls. Total viable FAPS (Viable, Ter119−/CD45/CD31−/integrin-α7−/sca1+) and macrophages (Viable, CD45+ F4/80+) counts were quantified using MACSQuantify software and normalized according to grams of muscle tissue. FlowJo v10 software was used for data analysis and data elaboration.

### Statistical Analysis

Results are expressed as mean±standard error of means (SEM). Differences among the groups were analysed using one-way ANOVA Test. When ANOVA revealed significant differences, the Tukey post hoc test was performed. The significance level was set at p<0.05. The statistical analyses were calculated using GraphPad Prism version 7 for Windows (San Diego, CA: GraphPad Software, Inc)

### Additional Information Regarding Human Tissue Procedure:

An open muscle biopsy of the left biceps was performed in all patients to obtain FAP cells. The muscle biopsy was obtained from a linear incision in the skin, following the longitudinal axis of the muscle fibers. Once fascia was reached, resection of the muscle fragment of interest was performed with a scalpel. Biopsy tissue was cleaned from fat tissue and cut at 1 mm² for explants.

Muscle explants were cultured and an immunomagnetic separation was done using specific anti-CD56 antibodies (MiltenyiBiotec, Bergisch-Gladbch, Germany). The negative fraction (CD56−) was cultured and their purity was verified by immunofluorescence (IF) and flow cytometry. IF was used to analyse the positive expression of fibroblast marker TE-7 and negative expression for desmin. Flow cytometry was used to analyse the expression of PDGFRa. Representative images of the IF and quantification of the PDGFRa expression is shown in FIGS. 1A and 1B. Histogram obtained from flow-cytometry experiments is shown in FIG. 1C. Stained cells were analysed by MACSQuant 10 (Miltenyi Biotec). Compensations were adjusted according to the single stained controls. FlowJo v10 software was used for data analysis and data elaboration.

### Additional Information Regarding Quantitative Proteomics:

### Protein Extraction, Quantification and Digestion

The protein samples were extracted with 6M Urea/200 mM Ammonium Bicarbonate (ABC) and precipitated with cold acetone. Cleaned samples were solubilized in the previous buffer described above and then quantified with the RCDC Protein Assay kit (Biorad, Hercules, CA). Ten μg of protein from each sample were digested in-solution using both Lys-C and Trypsin. Briefly, the samples were reduced with 10 mM dithiothreitol (DTT, in 200 mM ABC) for 1 h at 30° C. and 650 rpm in the thermo-mixer, alkylated with 20 mM chloroacetamide (CAA, in 200 mM ABC) for 30 minutes at room temperature in the dark at 650 rpm in the thermo-mixer. Then, samples were diluted to 2M Urea final concentration and the required amount of 1 μg/μl LysC (WAKO, Osaka, Japan) was added to obtain a 1:10 ratio enzyme: protein (w: w). The digestion was performed overnight at 37° C. at 650 rpm in the thermo-mixer. After that, samples were diluted at 1M Urea final concentration. Finally, the required amount of 1 μg/μl trypsin (Promega, Madison, WI) was added to obtain a 1:10 ratio enzyme: protein (w: w) and incubated for 8 h at 37° C. at 650 rpm in the thermo-mixer. Peptide mixtures were desalted using the commercial columns Ultra Microspin C18, 300A silica (The Nest Group, MA, USA) according to the manufacturer's instructions. Finally, the samples were dried in a SpeedVac and kept at −20° C. until the LC-MS/MS analysis.

### LC-MS/MS Analysis

The peptide samples were analysed using a Lumos Orbitrap mass spectrometer (Thermo Fisher Scientific, San Jose, CA, USA) coupled to an EASY-nLC 1000 (Thermo Fisher Scientific (Proxeon), Odense, Denmark). Peptide were loaded directly onto the analytical column and were separated by reversed-phase chromatography using a 50 cm column with an inner diameter of 75 μm, packed with 2 μm C18 particles spectrometer (Thermo Fisher Scientific) with a 110 min run, comprising consecutive steps with linear gradients from 5% to 22% B in 80 min, from 22% to 32% B in 10 min and from 32% to 95% B in 20 min at 250 nL/min flow rate and a binary solvent system of 0.1% formic acid in H2O (Solvent A) and 0.1% formic acid in acetonitrile (Solvent B). After each analysis, the column was washed for 10 min with 5% buffer A and 95% buffer B.

The mass spectrometer was operated in a data-dependent acquisition (DDA) mode and full MS scans with 1 micro scans at resolution of 120.000 were used over a mass range of m/z 350-1500 with detection in the Orbitrap. Auto gain control (AGC) was set to 2E5 and dynamic exclusion to 60 seconds. In each cycle of DDA analysis, following each survey scan Top Speed ions with charged 2 to 7 above a threshold ion count of 1e4 were selected for fragmentation at normalized collision energy of 28%. Fragment ion spectra produced via high-energy collision dissociation (HCD) were acquired in the Ion Trap, AGC was set to 3e4, isolation window of 1.6 m/z and maximum injection time of 40 ms was used. All data were acquired with Xcalibur software v3.0.63.

### Raw Data Processing and Database Search

The samples were analysed with the MaxQuant software (version 1.6.1.0) through the human Swissprot database. Trypsin was chosen as enzyme and a maximum of two missed cleavages were allowed. Carbamidomethylation (C) was set as a fixed modification, whereas oxidation (M) and acetylation (N-terminal) were used as variable modifications. Searches were performed using a peptide tolerance of 10 ppm and a product ion tolerance of 0.5 Da. Resulting data files were filtered for FDR<1% at both peptide and protein level. The non-unique peptides were assigned to the corresponding protein group according to the Razor peptides rule implemented in the software. The option “match between runs” was also enabled. The intensity values were normalized using the LFQ algorithm.

### Statistical and Bioinformatic Analysis

The final list of proteins was analysed using R. First, the protein list was filtered to remove the peptides/proteins tagged as “Reverse” (significantly identified in the reverse database), “potential contaminant” (items identified as contaminants in the “contaminants.fasta” file) and “Only identified by site” (proteins identified only with modified peptides). Then, the proteins with at least 75% of valid values in each experimental condition were selected. The missing values were imputed using the knn algorithm. The comparisons between groups were done using a t-test. The p-values were adjusted using the Benjamini-Hochberg procedure.

The total proteins analysed were represented in a volcano plot generated by Graphpad Prism Software. Proteomap was performed with upregulated proteins in PDGF-AA treated cells and was generated to visualise the differential contribution of biological pathways (bionic-vis.biologie.uni-greifswald.de/, v2.0). The map is created from the Kyoto Encyclopedia of Genes and Genomes (KEGG) functional classification and it shows the quantitative composition of proteomes. Each protein is represented by a polygon: areas reflect protein abundance, and functionally related proteins are arranged in common and similarly colored regions (34). The area for each protein reflects the magnitude of the fold change in PDGF-AA treated cells in comparison with untreated cells. The upregulated proteins represented in the proteomap were also analysed for enrichment of pathways in the Reactome database and were represented in a heatmap generated by the Clustvis online tool (biit.cs.ut.ee/clustvis/).

### Additional Information Regarding G-LISA, ROCK Activity Assay, Sircol and Cytokine Array: G-LISA

The RhoA-GTP protein was analysed by a colorimetric assay using the G-LISA assay (Cytoskeleton). The pathway was inhibited with the C3-exoenzyme (Cytoskeleton) inhibitor at 2 μg/ml overnight. Then, the pathway was activated with PDGF-AA at 50 ng/ml for 20 minutes at 37° C. and the G-LISA assay (Cytoskeleton) was performed following the manufacturer's instructions. The RhoA-GTP measurement was normalized to the amount of total RhoA that was measured by Western-blot.

### ROCK Activity Assay

The enzymatic activity of ROCK was assessed by a colorimetric method by using a ROCK activity assay (Abcam) in protein extract from quadriceps. The experiment was carried out according to the protocol provided by the manufacturers and the ROCK measurement was normalized to the amount of total protein used measured by Western-blot.

### Sircol

Total soluble collagen released by cultured FAPs was measured using a colorimetric assay. The test was carried out with the commercial Sircol kit, following the manufacturer's instructions (Biocolor, UK). The result was measured using the Coulter AD 340 plate reader (Beckam-Coulter, Brea, CA, USA) with the AD-LD program. At the end of the assay, the nuclei were stained with TO-PRO-3 (Thermo Fischer Scientific) and the signal emitted was measured with the Odyssey reader (LI-COR). The results obtained with the Sircol assay were normalized using the signal emitted by TO-PRO-3 to subtract the effect of the increased proliferation after PDGF-AA treatment. Representative image of the TO-PRO-3 staining is shown in supplementary FIG. 1G.

### Cytokine Array

Relative expression of proinflammatory cytokines were measured by Proteome Profiler Array, Panel A (R&D Systems, USA) using 100 μg of protein extract from quadriceps from mice and according to manufacturer's protocol. Three random samples from each mice group were used for the analysis. Quantitative analysis of blotting spot was performed using Image Studio 5.2 (LI-COR, Lincoln, NE). Relative results were normalized with the DBA/2J-WT group.

### Additional Information Regarding Proliferation and Migration Assays:

### Proliferation Assay

The proliferation assay was carried out following the manufacturer's instructions (Roche, Indianapolis, IN) at 24 and 72 hours. The result was measured using the Coulter AD 340 plate reader (Beckam-Coulter, Brea, CA, USA) with the AD-LD program.

### Migration Assay

Cells were plated in 24-well plates with inserts separating 2 chambers (Ibidi, Munich, Germany) and treated with 25 μg/ml of mitomycin C (Sigma-Aldrich, San Luis, MO, USA) for 1 hour. After treatment with the corresponding treatment at 48 and 72 hours, cells were fixed in 4% paraformaldehyde (PFA) and stained with Hoechst 33342. A total of 3 images were made at 10× of each well and the quantification of cells that migrated to the 500 μm space was quantified with Fiji software.

### Additional Information Regarding Western Blot

30 μg of total protein was loaded onto a 10% polyacrylamide gel and transferred to a nitrocellulose membrane. Nonspecific binding sites were blocked for 1 hour in casein diluted 1:1 with Tris-buffered saline (TBS). The membranes were incubated overnight with the primary antibody corresponding to each experimental condition. Corresponding secondary antibodies bound to Dye680 or Dye800 (Li-Cor) were incubated for one hour at 1:7,500 dilution (table 1).

### RNA Extraction, Reverse Transcription and Real-Time Quantitative PCR

Total RNA was extracted from quadriceps samples from mice using RNeasy Micro Kit (Qiagen, Hilden, Germany) following manufacturer's instructions. RNA was quantified using a nanodrop ND-1000 spectrophotometer (Nanodrop Technologies Inc., Wilmington, DE, USA). Five hundred ng of total RNA was reverse transcribed to complementary DNA (cDNA) using the High Capacity cDNA Reverse Transcription Kit (Applied Biosystems, Foster City, CA, USA). Real-Time PCR (qPCR) was performed using the TaqMan R Universal PCR Master Mix (Applied Biosystems, Foster City, CA, USA) and a 7900HT Fast Real-Time PCR System (Applied Biosystems, Foster City, CA). All mRNA-specific FAM-labelled primers/probe were purchased from Applied Biosystems. The mRNA-specific probes used are: Gapdh (Mm99999915_g1), Fhod1 (Mm00624707_m1), Rock2 (Mm01270843_m1) and Limk2 (Mm01187665_m1). Relative quantification was performed using the comparative Ct method and all results were compared with the control samples. GAPDH was used as endogenous control.

TABLE 1
Clinical information of DMD patients.

| | DMD patient 1 | DMD patient 2 | DMD patient 3 |
|---|---|---|---|
| Age | 9 yo | 9 yo | 7 yo |
| DMD mutations | 48-52 Deletion | 50-52 Deletion | 52 Deletion |
| Muscle biopsied | Biceps | Biceps | Biceps |
| Clinical status | Ambulant | Ambulant | Ambulant |
| Steroid treatment | Yes | Yes | Yes |

Yo = years old

TABLE 2
Primary, secondary antibodies and dyes used in this study.

| Target | Host | Dilution | Application | Manufacturer |
|---|---|---|---|---|
| ArHGEF2 | Rabbit | 1/100 | WB | Sigma-Aldrich |
| RhoA | Rabbit | 1/100 | WB | Cell Signalling |
| TE-7 | Mouse | 1/100 | IF | Merck |
| Desmin | Mouse | 1/50 | IF | Novocastra |
| p-MLC2 | Mouse | 1/100 | IF | Cell Signalling |
| PDGFRα | Goat | 1/50 | IF | R&D System |
| LIMK2 | Rabbit | 1/100 | IF | ProteinTech |
| PDGFA | Rabbit | 1/50 | IF | Merck Millipore |
| MLC2 | Rabbit | 1/50 | IF | Cell Signalling |
| F4/80 | Rabbit | 1/200 | IF | Cell Signalling |
| CD3 | Rabbit | 1/50 | IF | Abcam |
| Ly6g + Ly6c | Rat | 1/50 | IF | Abcam |
| CD19 | Rabbit | 1/50 | IF | Abcam |
| Wheatgerm | N/A | 1/100 | IF | Invitrogen |
| agglutinin-488¹ | | | | |
| Hoesch 33342¹ | N/A | 1/1000 | IF | Invitrogen |
| Rhodamine | N/A | 1/1000 | IF | Abcam |
| Phalloidin¹ | | | | |
| CD140a-PE | Recombinant | 1/200 | IF | Milteny Biotech |
| CD56 | Ms | N/A | MACS | Miltenyi Biotech |
| PDGFRα-biot | Goat | 1/75 | FC | R&D System |
| CD56-PE | Ms | 1/20 | FC | Miltenyi Biotech |
| LIVE/DEAD¹ | N/A | 1/1000 | FC | Thermo Fisher |
| F4/80 | Rat | N/A | FC | Bio Legend |
| CD45 | Rat | N/A | FC | eBioscience |
| Integrin- α7 | Rat | N/A | FC | Milteny Biotec |
| Ter119 | Rat | N/A | FC | eBioscience |
| CD31 | Rat | N/A | FC | Thermo Fisher |
| Sca1 | Rat | N/A | FC | BD |
| CD163 | Rat | N/A | FC | eBioscience |
| CD206 | Rat | N/A | FC | Bio Legend |
| CD80 | REA | N/A | FC | Milteny Biotec |
| HLA-DR | Rat | N/A | FC | Milteny Biotec |
| Secondary | Dilution | Conjugatio | Application | Manufacturer |
| Donkey anti-goat | 1/500 | Alexa594 | IF | Invitrogen |
| Donkey anti-rabbit | 1/500 | Alexa647 | IF | Invitrogen |
| Goat anti-mouse | 1/500 | Alexa488 | IF | Invitrogen |
| Goat anti-rabbit | 1/500 | Alexa488 | IF | Invitrogen |
| Goat anti-rat | 1/500 | Alexa594 | IF | Invitrogen |
| Goat anti-rabbit | 1/7500 | IRDye 800 | WB | LICOR |
| Goat anti-rabbit | 1/2000 | N/A | WB | Invitrogen |
| biotinilyated | | | | |
| Streptavidin 800 | 1/2000 | IRDye 800 | WB | LICOR |
| Streptavidin PE-Cy5 | 1/200 | PE-Cy5 | FC | Biolegend |

¹It is not an antibody.
²Volume per test following manufacturer's instructions.
N/A: not applicable;
Ms: Mouse;
IHQ: Immunohistochemistry;
FC: Flow cytometry.
MACS: Magnetic activated cell sorting

### Sequences:

MLCK inhibitor peptide 18
(SEQ ID NO: 1):
RKKYKYRRK

1. A method of treating a disease or disorder associated with muscle fibrosis in a subject, the method comprising administering a therapeutically effective amount of a RhoA/ROCK pathway inhibitor to the subject.

2. The method according to claim 1, wherein the disease or disorder associated with muscle fibrosis is a muscular dystrophy.

3. The method according to claim 1, wherein the disease or disorder associated with muscle fibrosis is a dystrophinopathy.

4. The method according to claim 1, wherein the disease or disorder associated with muscle fibrosis is selected from the group consisting of: Duchenne muscular dystrophy, Becker muscular dystrophy, recessive and dominant limb girdle muscular dystrophy, myotonic dystrophy type I, myotonic dystrophy type II, facio-scapulo-humeral muscular dystrophy, congenital muscular dystrophy, oculo-pharyngeal muscular dystrophy, Emery-Dreifuss muscular dystrophy, inclusion body myositis, distal myopathy with dystrophic changes and myofibrillar myopathy.

5. The method according to claim 1, wherein the inhibitor inhibits the RhoA/ROCK pathway by directly inhibiting ROCK1 and/or ROCK2.

6. The method according to claim 1, wherein the inhibitor is selected from the group consisting of: Fasudil, Y27632, Y39983, Wf-536, AR-13324, AR-12286, AMA0076, PG324, Azabenzimidazole-aminofurazans, DE-104, Olefins, Isoquinolines, Indazoles, pyridinealkene derivates, H-1152P, ROKα inhibitor, XD-4000, HMN-1152, 4-(1-aminoalkyl)-N-(4-pyridyl)cyclohexane-carboxamides, Quinazoline, Ripasudil, VAS-012, Ki-23095, BA-2017, BA-215, BA-285, BA-1037, BA-210, Rhostatin, ROCK-IN-1—preclinical, ROCK inhibitor-2, ROCK2-IN-5, ROCK2-IN-2, ROCK-IN-2 (Azaindole 1; TC-S 7001), Chroman 1, Cotosudil, SAR407899 hydrochloride, SAR407899, Hydroxyfasudil, Ripasudil, Ripasudil free base, Belumosudil mesylate, Belumosudil, Sovesudil, RKI-1447, H-1152, GSK269962A, Rho-Kinase-IN-1, SB-772077B dihydrochloride, HSD1590, BDP5290, ZINC00881524, Verosudil, LX7101, GSK-25, GSK180736A, CRT0066854, Y-27632, CMPD101, Thiazovivin, SR-3677, and GSK429286A, or pharmaceutically acceptable salts, solvates or prodrugs thereof.

7. The method according to claim 1, wherein the inhibitor is a compound of formula (1) or a pharmaceutically acceptable salt, solvate or prodrug thereof: 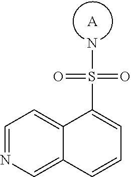
wherein:
A is a monocyclic 5- to 7-membered heterocycloalkyl ring optionally substituted with one or more R¹;
each R¹ is independently selected from C₁₋₆alkyl, hydroxy-C₁₋₆alkyl, halo-C₁₋₆alkyl and —C(O)OR²; and
R² is C₁₋₆ alkyl, and
optionally wherein the compound of formula (1) is Fasudil.

8. The method according to claim 1, wherein the inhibitor is selected from the group consisting of: C3 exoenzyme, C3 Trans based, Rhosin, CCG-1423, CCG-203971, YS-49 monohydrate, YS-49, Cerivastatin sodium, Cerivastatin, Z62954982, Y16, MLS-573151, HA-100, HL07, DDO-5701, DDO-5713, DDO-5714, DDO-5715, DDO-5716, ML-7, MLCK18, and CT-04 or pharmaceutically acceptable salts, solvates or prodrugs thereof.

9. The method according to claim 8, wherein the inhibitor inhibits the RhoA/ROCK pathway by ribosylating RhoA proteins.

10. The method according to claim 9, wherein the inhibitor is C3 exoenzyme.

11. Use of a RhoA/ROCK pathway inhibitor for treating a disease or disorder associated with muscle fibrosis.

12. The use according to claim 11, wherein the disease or disorder associated with muscle fibrosis is a muscular dystrophy.

13. The use according to claim 11, wherein the disease or disorder associated with muscle fibrosis is a dystrophinopathy.

14. The use according to claim 11, wherein the disease or disorder associated with muscle fibrosis is selected from the group consisting of: Duchenne muscular dystrophy, Becker muscular dystrophy, recessive and dominant limb girdle muscular dystrophy, myotonic dystrophy type I, myotonic dystrophy type II, facio-scapulo-humeral muscular dystrophy, congenital muscular dystrophy, oculo-pharyngeal muscular dystrophy, Emery-Dreifuss muscular dystrophy, inclusion body myositis, distal myopathy with dystrophic changes and myofibrillar myopathy.

15. The use according to claim 11, wherein the inhibitor inhibits the RhoA/ROCK pathway by directly inhibiting ROCK1 and/or ROCK2.

16. The use according to claim 11, wherein the inhibitor is selected from the group consisting of: Fasudil, Y27632, Y39983, Wf-536, AR-13324, AR-12286, AMA0076, PG324, Azabenzimidazole-aminofurazans, DE-104, Olefins, Isoquinolines, Indazoles, pyridinealkene derivates, H-1152P, ROKα inhibitor, XD-4000, HMN-1152, 4-(1-aminoalkyl)-N-(4-pyridyl)cyclohexane-carboxamides, Quinazoline, Ripasudil, VAS-012, Ki-23095, BA-2017, BA-215, BA-285, BA-1037, BA-210, Rhostatin, ROCK-IN-1—preclinical, ROCK inhibitor-2, ROCK2-IN-5, ROCK2-IN-2, ROCK-IN-2 (Azaindole 1; TC-S 7001), Chroman 1, Cotosudil, SAR407899 hydrochloride, SAR407899, Hydroxyfasudil, Ripasudil, Ripasudil free base, Belumosudil mesylate, Belumosudil, Sovesudil, RKI-1447, H-1152, GSK269962A, Rho-Kinase-IN-1, SB-772077B dihydrochloride, HSD1590, BDP5290, ZINC00881524, Verosudil, LX7101, GSK-25, GSK180736A, CRT0066854, Y-27632, CMPD101, Thiazovivin, SR-3677, and GSK429286A, or pharmaceutically acceptable salts, solvates or prodrugs thereof.

17. The use according to claim 11, wherein the inhibitor is a compound of formula (1) or a pharmaceutically acceptable, salt, solvate or prodrug thereof: 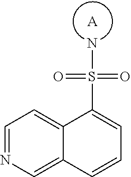
wherein:
A is a monocyclic 5- to 7-membered heterocycloalkyl ring optionally substituted with one or more R¹;
each R¹ is independently selected from C₁₋₆alkyl, hydroxy-C₁₋₆alkyl, halo-C₁₋₆alkyl and —C(O)OR²; and
R² is C₁₋₆ alkyl, and
optionally wherein the compound of formula (1) is Fasudil.

18. The use according to claim 11, wherein the inhibitor is selected from the group consisting of C3 exoenzyme, C3 Trans based, Rhosin, CCG-1423, CCG-203971, YS-49 monohydrate, YS-49, Cerivastatin sodium, Cerivastatin, Z62954982, Y16, MLS-573151, HA-100, HL07, DDO-5701, DDO-5713, DDO-5714, DDO-5715, DDO-5716, ML-7, MLCK18, CT-04 or pharmaceutically acceptable salts, solvates or prodrugs thereof.

19. The use according to claim 18, wherein the inhibitor inhibits the RhoA/ROCK pathway by ribosylating RhoA proteins.

20. The use according to claim 19, wherein the inhibitor is C3 exoenzyme.


SEQUENCE LISTING (1 sequence)

SEQ ID NO: 1 - protein, 9 aa, Artificial Sequence
  MLCK inhibitor peptide 18
RKKYKYRRK